SEQUENCE LISTING (133 sequences)

SEQ ID NO: 1 - RNA, 19 nt, synthetic construct
  source 1..19: mol_type = other RNA
nrndsassancasssnnyn

SEQ ID NO: 2 - RNA, 27 nt, synthetic construct
  source 1..27: mol_type = other RNA
ttctccacatgaggatcacccatgtgg

SEQ ID NO: 3 - RNA, 19 nt, synthetic construct
  source 1..19: mol_type = other RNA
gggccctgaagaagggccc

SEQ ID NO: 4 - RNA, 33 nt, synthetic construct
  source 1..33: mol_type = other RNA
ggatagggccctgaagaagggccctatctcttc

SEQ ID NO: 5 - RNA, 25 nt, synthetic construct
  source 1..25: mol_type = other RNA
ggagcagacgatatggcgtcgctcc

SEQ ID NO: 6 - RNA, 27 nt, synthetic construct
  source 1..27: mol_type = other RNA
ccagatctgagcctgggagctctctgg

SEQ ID NO: 7 - RNA, 12 nt, synthetic construct
  source 1..12: mol_type = other RNA
gttttagagcta

SEQ ID NO: 8 - RNA, 12 nt, synthetic construct
  source 1..12: mol_type = other RNA
gtttaagagcta

SEQ ID NO: 9 - RNA, 12 nt, synthetic construct
  source 1..12: mol_type = other RNA
gtttaagagata

SEQ ID NO: 10 - RNA, 13 nt, synthetic construct
  source 1..13: mol_type = other RNA
gtttaagagataa

SEQ ID NO: 11 - RNA, 12 nt, synthetic construct
  source 1..12: mol_type = other RNA
gtttaagagtta

SEQ ID NO: 12 - RNA, 12 nt, synthetic construct
  source 1..12: mol_type = other RNA
gtttaagagttc

SEQ ID NO: 13 - RNA, 12 nt, synthetic construct
  source 1..12: mol_type = other RNA
gtttaagagcgc

SEQ ID NO: 14 - RNA, 13 nt, synthetic construct
  source 1..13: mol_type = other RNA
gtttaagagcgtc

SEQ ID NO: 15 - RNA, 14 nt, synthetic construct
  source 1..14: mol_type = other RNA
gtttaagagcgtac

SEQ ID NO: 16 - RNA, 15 nt, synthetic construct
  source 1..15: mol_type = other RNA
gtttaagagcgtagc

SEQ ID NO: 17 - RNA, 16 nt, synthetic construct
  source 1..16: mol_type = other RNA
gtttaagagcgtagcg

SEQ ID NO: 18 - RNA, 17 nt, synthetic construct
  source 1..17: mol_type = other RNA
gtttaagagcgtagctg

SEQ ID NO: 19 - RNA, 18 nt, synthetic construct
  source 1..18: mol_type = other RNA
gtttaagagcgtcagctg

SEQ ID NO: 20 - RNA, 60 nt, synthetic construct
  source 1..60: mol_type = other RNA
gcgcaagtttaaataaggctagtccgttatcaacttgaaaaagtgggaccgagtcggtcc

SEQ ID NO: 21 - RNA, 14 nt, synthetic construct
  source 1..14: mol_type = other RNA
tagcaagtttaaat

SEQ ID NO: 22 - RNA, 14 nt, synthetic construct
  source 1..14: mol_type = other RNA
tatcaagtttaaat

SEQ ID NO: 23 - RNA, 15 nt, synthetic construct
  source 1..15: mol_type = other RNA
ttatcaagtttaaat

SEQ ID NO: 24 - RNA, 14 nt, synthetic construct
  source 1..14: mol_type = other RNA
taacaagtttaaat

SEQ ID NO: 25 - RNA, 14 nt, synthetic construct
  source 1..14: mol_type = other RNA
gaacaagtttaaat

SEQ ID NO: 26 - RNA, 14 nt, synthetic construct
  source 1..14: mol_type = other RNA
gcgcaagtttaaat

SEQ ID NO: 27 - RNA, 15 nt, synthetic construct
  source 1..15: mol_type = other RNA
gacgcaagtttaaat

SEQ ID NO: 28 - RNA, 16 nt, synthetic construct
  source 1..16: mol_type = other RNA
gtacgcaagtttaaat

SEQ ID NO: 29 - RNA, 17 nt, synthetic construct
  source 1..17: mol_type = other RNA
gctacgcaagtttaaat

SEQ ID NO: 30 - RNA, 18 nt, synthetic construct
  source 1..18: mol_type = other RNA
cgctacgcaagtttaaat

SEQ ID NO: 31 - RNA, 19 nt, synthetic construct
  source 1..19: mol_type = other RNA
cagctacgcaagtttaaat

SEQ ID NO: 32 - RNA, 20 nt, synthetic construct
  source 1..20: mol_type = other RNA
cagctgacgcaagtttaaat

SEQ ID NO: 33 - RNA, 46 nt, synthetic construct
  source 1..46: mol_type = other RNA
aaggctagtccgttatcaacttgaaaaagtgggaccgagtcggtcc

SEQ ID NO: 34 - RNA, 26 nt, synthetic construct
  source 1..26: mol_type = other RNA
tctgccatcaaagcgtgctcagtctg

SEQ ID NO: 35 - RNA, 42 nt, synthetic construct
  source 1..42: mol_type = other RNA
ttgacgcggttctatctagttacgcgttaaaccaactagaaa

SEQ ID NO: 36 - RNA, 101 nt, synthetic construct
  source 1..101: mol_type = other RNA
ggcccagactgagcacgtgagtttaagagcgcttctccacatgaggatcacccatgtggttgacgcggttctatctagttacgcgttaaaccaactagaaa

SEQ ID NO: 37 - RNA, 161 nt, synthetic construct
  source 1..161: mol_type = other RNA
ggatagggccctgaagaagggccctatctcttcgcgcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 38 - DNA, 2064 bp, synthetic construct
  source 1..2064: mol_type = other DNA
atgcgggaccacatggtgctgcacgagagcgtgaacgccgccggcatcacctctggcggctcaaaaagaaccgccgacggcagcgaattcgagtcacccaagaagaagaggaaagtcggcggccctgctgccaagagggtcaagttggacggaagcggagccagcaatttcacccagttcgtgctggtcgacaacggcggtacaggcgatgtgaccgtggcccctagcaacttcgccaacggcgtggccgagtggatcagcagcaacagcagaagccaggcctataaggtgacatgcagcgtgcggcagtcttctgcccagaagcgcaagtacaccatcaaggtggaggtgcctaaagtggctacccaaacagtgggaggcgtggagctgcctgtggcagcctggcggagctacctgaacatggaactcaccatccctatcttcgccacgaacagcgactgtgagctgatcgtgaaagccatgcagggcctgctgaaggacggcaaccccatcccttctgccatcgccgctaatagtggactgtactaatgaccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccccaaagctggctacaatggagcagaagctgatcagcgaggaagatctgaagcgccccgccgctacaaagaaagccggccaggccaagaagaagaaaggcggttccgccagcggcggcagcatggacgcccaaaccaggagacgggaacggagagccgagaagcaggctcagtggaaggccgctaattctggaggatctagcggaggatccaaacggacagccgacggaagcgagttcgagtcaccaaagaagaagcggaaagtctctggcggttcaagcggcggctctgagttcatgagcaagggcgaggaactgttcaccggagtggtgccaatcctggtggaactggacggcgacgtgaacggccacaagttcagcgtcagaggcgaaggagagggcgacgccacaatcggcaagctgaccctgaagtttatctgcaccaccggcaagctccccgtgccctggcccaccctggtgacaaccctgacatacggcgttcaatgttttagcagataccccgatcacatgaaaaggcacgacttcttcaagtccgccatgcctgagggctacgtgcaggagcggaccatcagctttaaggacgacggcaaatacaagacaagagccgtggtcaagttcgagggcgacaccctggttaatagaatcgagctgaagggcactgatttcaaggaggacggcaacatcctgggccacaagctggaatacaacttcaacagccacaacgtgtacatcacagctgacaagcagaagaacggcatcaaagccaatttcaccgtgcggcacaacgtggaagatggcagcgtgcagctggccgatcattatcagcagaacacccctattggcgatggacctgtgctgctgcctgacaaccactacctgtccacccaaaccgtgctgagcaaggaccccaacgagaagggaaca

SEQ ID NO: 39 - DNA, 8718 bp, synthetic construct
  source 1..8718: mol_type = other DNA
atgaaacggacagccgacggaagcgagttcgagtcaccaaagaagaagcggaaagtcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagaaagctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaagagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactccggcggaagctctggtggcagcaagcggaccgccgacggctctgaattcgagagccctaagaagaaaagaaaggtgagcggaggctctagcggcggaagcaccctgaacattgaagacgagtatagactgcatgaaacaagcaaggaacccgacgtgtccctgggctccacctggctgtccgactttccccaggcctgggccgagacaggaggaatgggcctggccgtgcggcaggcacccctgatcatccctctgaaggccacctctacacccgtgagcatcaagcagtaccctatgtctcaggaggccagactgggcatcaagcctcacatccagaggctgctggaccagggcatcctggtgccatgccagagcccctggaacacaccactgctgcccgtgaagaagccaggcaccaatgactatagacccgtgcaggatctgagagaggtgaacaagagggtggaggatatccaccccaccgtgcccaacccttacaatctgctgtccggcctgcccccttctcaccagtggtatacagtgctggacctgaaggatgccttcttttgtctgagactgcaccctaccagccagccactgttcgcctttgagtggagggaccctgagatgggcatctctggccagctgacctggacacgcctgcctcagggcttcaagaatagcccaacactgtttaacgaggccctgcaccgcgacctggcagatttccggatccagcacccagatctgatcctgctgcagtacgtggacgatctgctgctggccgccaccagcgagctggattgccagcagggaacacgcgccctgctgcagaccctgggaaacctgggatatagggcatccgccaagaaggcccagatctgtcagaagcaggtgaagtacctgggctatctgctgaaggagggccagagatggctgacagaggccaggaaggagacagtgatgggccagccaacacccaagaccccaagacagctgagggagttcctgggcaaagcaggattttgcaggctgttcatcccaggattcgcagagatggcagcacctctgtacccactgaccaagccgggcaccctgtttaattggggccctgaccagcagaaggcctatcaggagatcaagcaggccctgctgacagcaccagccctgggcctgccagacctgaccaagcctttcgagctgtttgtggatgagaagcagggctacgccaagggcgtgctgacccagaagctgggaccatggagacggcccgtggcctatctgtccaagaagctggacccagtggcagcaggatggccaccatgcctgaggatggtggcagcaatcgccgtgctgacaaaggatgccggcaagctgaccatgggacagccactggtcatcctggcaccacacgcagtggaggccctggtgaagcagcctccagatcgctggctgtctaacgcccggatgacacactaccaggccctgctgctggacaccgatcgcgtgcagtttggccctgtggtggccctgaatccagccaccctgctgcctctgccagaggagggcctgcagcacaactgtctggacatcctggcagaggcacacggaacaaggccagacctgaccgatcagcccctgcctgacgccgatcacacatggtataccgatggaagctccctgctgcaggagggccagaggaaggcaggagcagcagtgaccacagagacagaagtgatctgggccaaggccctgccagcaggcacatccgcccagcgggccgagctgatcgccctgacccaggccctgaagatggccgagggcaagaagctgaacgtgtacacagactccagatatgccttcgccaccgcacacatccacggagagatctacaggcgccggggctggctgacctctgagggcaaggagatcaagaacaaggatgagatcctggccctgctgaaggccctgtttctgcccaagcggctgagcatcatccactgtcctggacaccagaagggacactccgccgaggcaaggggcaatcggatggccgaccaggccgccagaaaggctgctattactgaaactcccgacacttccactctgctgattgaaaactcctccccttctggcggctcaaaaagaaccgccgacggcagcgaattcgagtctcccaagaagaagaggaaagtcggctctggccctgccgctaagagagtgaagctggacggaagcggagctactaacttcagcctgctgaagcaggctggagacgtggaggagaaccctggacctagcttcgttgctggagtcatccggagactggacgagacagtggtgaacagaattgccgccggcgaggtgatccagagacctgccaatgcaataaaggagatgatcgagaactgtctggacgccaagtccacaagcattcaggtgatcgtgaaggagggcggactgaagctgatccagatccaagacaacggcacaggcatcagaaaggaagatctggacatcgtgtgtgaacggttcaccacatctaagctgcagtcttttgaggatctggcctctatcagtacctacggcttcagaggcgaggccctggccagcatcagccacgtggcccatgtgaccatcaccaccaaaaccgccgacggcaaatgcgcttatcgcgctagctacagcgacggcaagctgaaagccccgccaaagccttgcgccggcaaccagggtacacagataacagtggaggatctgttctacaacatcgccacccggagaaaggccctgaaaaatcccagcgaggagtacggcaagatcctggaagtcgtcggcagatactccgtgcacaacgccggaatcagctttagcgtaaagaagcagggagaaaccgtggccgatgtgcgcaccctgccaaatgccagcaccgtggataacatcagaagcattttcggaaatgccgtgtccagagaactgatcgagatcggctgcgaagataagaccctggcttttaagatgaacggctacatcagcaacgccaattactctgtgaagaagtgcatctttcttctgttcatcaaccacagactggtggaaagcaccagcctgcggaaagccatcgagacagtgtacgccgcctacctgcctaagaacacccaccccttcctgtacctgagcctcgagatcagccctcagaacgtggacgtcaatgtgcatcctacaaagcacgaggtgcacttcctgcacgaggaaagcatcctggaaagagtgcagcagcacattgagagcaagctgctgggctctaacagcagcagaatgtacttcacacagaccctgctgcctggcctggccggcccctcaggcgaaatggttaagtccacaacctctctgacctcatccagcaccagcggttcttccgataaggtgtacgcccaccagatggtgcggaccgactctcgggagcagaagctggacgcctttctgcaacctctgagcaaacctctgagctctcagcctcaggccatcgtgaccgaggacaagacagatatctcctccggccgtgccagacagcaggacgaagaaatgctcgagctgccagctcctgccgaggtggccgccaagaaccagagcctggagggagataccacaaagggcaccagcgaaatgagcgagaagcggggccctacctccagcaaccccagaaaacggcaccgggaggacagcgacgtggaaatggtggaggacgacagcagaaaggaaatgacagccgcttgtacccctagaagaagaatcatcaacctgacctccgtgctgagcctgcaggaggagatcaacgagcagggccacgaggtgctgagagagatgctgcacaatcacagcttcgtgggctgcgtgaaccctcaatgggccctggctcagcatcaaacaaagctgtacctgctgaacaccaccaagctgagcgaagagctgttctaccagatcctcatctacgacttcgccaacttcggcgtgctacgcctgagcgagcccgcccctctgtttgacctggccatgctggctctggatagcccagaaagcggctggacagaagaggacggacctaaagaggggctggctgaatacatcgtggagttcctgaagaaaaaggccgagatgctggccgactacttttctctggaaatcgacgaggaaggcaacctgatcggcctgcctctgctgatcgataactacgtgcctcccctggaaggcctgcccatcttcatcctgagactggctacagaggtgaactgggacgaggaaaaggaatgcttcgagtctctgagcaaggagtgcgccatgttctatagcatcagaaaacagtacatctctgaagagagcactctgtctggccagcagagtgaagtgcccggaagcatccccaacagctggaagtggaccgtggaacacatcgtgtacaaggccctgcggagccacattctccctcctaagcacttcaccgaggacggcaacatcctgcagctggccaacctgcccgacctttataaggttttctaa

SEQ ID NO: 40 - DNA, 879 bp, synthetic construct
  source 1..879: mol_type = other DNA
atggccccaaagctggctacaggcggttccgccagcggcggcagcatggacgcccaaaccaggagacgggaacggagagccgagaagcaggctcagtggaaggccgctaattctggaggatctagcggaggatccatgggccctgatatcgtgatgacccagagccctagctccctgtctgcttctgtgggagatagagtgaccattacatgtagaagctccaccggcgccgtgacaaccagcaactacgcctcttgggtccaggagaagcctggcaaactgtttaagggcctgatcggaggaacaaacaacagagccccaggcgtccccagccggttcagcggcagcctgatcggcgataaggccacactgaccatcagcagcctgcagcctgaggacttcgccacctacttctgcgccctgtggtacagcaatcactgggtgttcggccagggcaccaaggtggaactgaagcggggtggaggtggttctggcggtggcgggtcaggaggcggaggatctagtggtgggggatccgaagtaaagttgttggagtccggtggtggcctggtgcagcccggcggcagcctgaaactgagctgcgctgtgtctggattttctctgacagattacggcgtgaactgggttaggcaggcccctggaagaggcctggaatggatcggcgttatctggggcgacggcatcaccgactacaacagcgccctgaaagacagattcatcatcagcaaggacaatggcaagaacaccgtgtacctgcaaatgagcaaggtgcggagcgacgacaccgccctgtactactgcgtgacaggactgttcgactattggggacagggcaccctcgtgaccgtgtccagctaa

SEQ ID NO: 41 - DNA, 468 bp, synthetic construct
  source 1..468: mol_type = other DNA
atgggaggagaagaacttttgagcaagaattatcatcttgagaacgaagtggctcgtcttaagaaatcaggaagcggagccagcaatttcacccagttcgtgctggtcgacaacggcggtacaggcgatgtgaccgtggcccctagcaacttcgccaacggcgtggccgagtggatcagcagcaacagcagaagccaggcctataaggtgacatgcagcgtgcggcagtcttctgcccagaagcgcaagtacaccatcaaggtggaggtgcctaaagtggctacccaaacagtgggaggcgtggagctgcctgtggcagcctggcggagctacctgaacatggaactcaccatccctatcttcgccacgaacagcgactgtgagctgatcgtgaaagccatgcagggcctgctgaaggacggcaaccccatcccttctgccatcgccgctaatagtggactgtactaa

SEQ ID NO: 42 - DNA, 540 bp, synthetic construct
  source 1..540: mol_type = other DNA
atggccccaaagctggctacaggcggttccgccagcggcggcagcatggacgcccaaaccaggagacgggaacggagagccgagaagcaggctcagtggaaggccgctaattctggaggatctagcggaggatcctctggcgaagtgcagctgcaggagagcggcggaggcctggtgcaacctggaggcagcctgagactgagctgcaccgccagcggcgtgaccatctctgctctgaacgccatggccatgggctggtatagacaggccccaggcgagcggcgggtgatggtcgccgctgtgtccgagcgcggaaatgccatgtaccgggaaagcgtgcagggcagattcaccgttacaagagattttacaaacaagatggtgtctctccagatggacaacctgaagcccgaggacaccgccgtgtactactgtcacgtgctggaagatagagtggacagcttccacgactactggggccagggcacccaggtgacagtgtccagcggcgcccctggcttcagcagcatcagcgcctaa

SEQ ID NO: 43 - DNA, 468 bp, synthetic construct
  source 1..468: mol_type = other DNA
atgggaggacccagcagactggaagaggaactgagacggagactgaccgagcctggctctggcggctcaggaagcggagccagcaatttcacccagttcgtgctggtcgacaacggcggtacaggcgatgtgaccgtggcccctagcaacttcgccaacggcgtggccgagtggatcagcagcaacagcagaagccaggcctataaggtgacatgcagcgtgcggcagtcttctgcccagaagcgcaagtacaccatcaaggtggaggtgcctaaagtggctacccaaacagtgggaggcgtggagctgcctgtggcagcctggcggagctacctgaacatggaactcaccatccctatcttcgccacgaacagcgactgtgagctgatcgtgaaagccatgcagggcctgctgaaggacggcaaccccatcccttctgccatcgccgctaatagtggactgtactaa

SEQ ID NO: 44 - DNA, 555 bp, synthetic construct
  source 1..555: mol_type = other DNA
atggagcagaagctgatcagcgaggaagatctgaagcgccccgccgctacaaagaaagccggccaggccaagaagaagaaaggcggttccgccagcggcggcagcatggacgcccaaaccaggagacgggaacggagagccgagaagcaggctcagtggaaggccgctaattctggaggatctagcggaggatccaaacggacagccgacggaagcgagttcgagtcaccaaagaagaagcggaaagtctctggcggttcaagcggcggctctatcctgtggcatgagatgtggcacgagggcctggaagaggcctctagactgtatttcggcgagcggaacgtcaagggaatgttcgaggtgctggaaccactgcacgccatgatggaaagaggccctcagaccctgaaggaaaccagctttaaccaggcctacggcagagatctgatggaagctcaggagtggtgcagaaagtacatgaaaagcggcaacgtgaaggacctgacacaggcctgggacctctactaccacgtgttcagaagaatctctaagtaa

SEQ ID NO: 45 - DNA, 822 bp, synthetic construct
  source 1..822: mol_type = other DNA
atgggcgtccaggtggaaaccatcagccctggagatggcagaaccttccccaagcggggccagacctgcgtggtgcactacacaggcatgctggaagatggaaagaaatttgacagctccagagatagaaacaagccttttaagttcatgctgggcaagcaggaggtgatcagaggctgggaggaaggcgttgctcagatgagcgtgggccaaagagccaagctgaccatttctcccgactacgcctacggcgccacaggccaccccggaatcatcccacctcacgccaccctggtgttcgacgtggagctgctgaaactggaatctggcggctcaaaaagaaccgccgacggcagcgaattcgagtcacccaagaagaagaggaaagtcggcggccctgctgccaagagggtcaagttggacggaagcggagccagcaatttcacccagttcgtgctggtcgacaacggcggtacaggcgatgtgaccgtggcccctagcaacttcgccaacggcgtggccgagtggatcagcagcaacagcagaagccaggcctataaggtgacatgcagcgtgcggcagtcttctgcccagaagcgcaagtacaccatcaaggtggaggtgcctaaagtggctacccaaacagtgggaggcgtggagctgcctgtggcagcctggcggagctacctgaacatggaactcaccatccctatcttcgccacgaacagcgactgtgagctgatcgtgaaagccatgcagggcctgctgaaggacggcaaccccatcccttctgccatcgccgctaatagtggactgtactaa

SEQ ID NO: 46 - DNA, 1038 bp, synthetic construct
  source 1..1038: mol_type = other DNA
atggccccaaagctggctacaggcggttccgccagcggcggcagcatggacgcccaaaccaggagacgggaacggagagccgagaagcaggctcagtggaaggccgctaattctggaggatctagcggaggatccacaagagtgcctctgtacggcttcaccagcatctgcggacggcgccccgagatggaagccgccgtttctaccatcccaagatttctgcagagcagctccggcagcatgctggacggcagattcgacccccagtccgccgcacatttcttcggcgtgtacgacggccacggcggctctcaggtggctaattactgcagagagcggatgcacctggccctggccgaggaaatcgccaaggagaagcccatgctctgtgacggagatacatggctggaaaagtggaagaaggccctgttcaacagcttcctgagagttgacagcgagatcgagagcgtggcccctgaaaccgtgggcagcacctccgtggtggctgtggtctttcccagccacatcttcgtggccaactgcggcgattctcgggccgtgctgtgtagaggcaagacagccctgcctctgtccgtggaccacaaacctgaccgggaagatgaggccgcccggatcgaggccgctggtggaaaggtgatccagtggaacggcgccagggtgttcggcgtgctggccatgagcagaagcatcggcgacagatatctgaaacctagcattatccctgatccagaggtgaccgccgtcaagcgggtgaaggaagatgactgcctgatcctggcttctgatggcgtgtgggacgtgatgaccgacgaggaggcctgcgagatggccagaaagagaatcctgctgtggcacaagaaaaacgccgtggccggcgacgccagcctgctggctgatgagagaagaaaggaaggcaaagaccctgccgctatgagcgccgctgaatacctgagcaagctggccatccaaagaggatctaaggacaacatcagcgtggtggtggtggacctgaagtaa

SEQ ID NO: 47 - DNA, 957 bp, synthetic construct
  source 1..957: mol_type = other DNA
atgggagctcctacccaagacgagttcacccagctgagccagagcatcgccgagtttcacacataccagctgggcaacggcagatgttccagcctgctggcccagagaatccacgcccctccagaaaccgtgtggagcgtggtgcggaggttcgacagaccccagatctataagcacttcatcaagagctgcaacgtgtccgaggacttcgagatgagagtgggctgcacacgggacgtgaacgtgatcagcggcctgcctgccaataccagcagagagcggctggatctgctggacgatgaccggcgggtgacaggcttcagcatcaccggaggcgagcaccggctcagaaactacaagtctgtgaccaccgtgcatagatttgagaaagaggaagaagaggaaagaatctggaccgtcgtcctggaaagctacgtggttgacgtgcccgagggcaattctgaagaagatacaagactgttcgccgataccgtgatcagactgaacctgcagaagctggcttctattacagaggccatgaacggctctggcggctcaggaagcggagccagcaatttcacccagttcgtgctggtcgacaacggcggtacaggcgatgtgaccgtggcccctagcaacttcgccaacggcgtggccgagtggatcagcagcaacagcagaagccaggcctataaggtgacatgcagcgtgcggcagtcttctgcccagaagcgcaagtacaccatcaaggtggaggtgcctaaagtggctacccaaacagtgggaggcgtggagctgcctgtggcagcctggcggagctacctgaacatggaactcaccatccctatcttcgccacgaacagcgactgtgagctgatcgtgaaagccatgcagggcctgctgaaggacggcaaccccatcccttctgccatcgccgctaatagtggactgtactaa

SEQ ID NO: 48 - DNA, 27 bp, synthetic construct
  source 1..27: mol_type = other DNA
ttctccacatgaggatcacccatgtgg

SEQ ID NO: 49 - DNA, 19 bp, synthetic construct
  source 1..19: mol_type = other DNA
gggccctgaagaagggccc

SEQ ID NO: 50 - DNA, 33 bp, synthetic construct
  source 1..33: mol_type = other DNA
ggatagggccctgaagaagggccctatctcttc

SEQ ID NO: 51 - DNA, 25 bp, synthetic construct
  source 1..25: mol_type = other DNA
ggagcagacgatatggcgtcgctcc

SEQ ID NO: 52 - DNA, 27 bp, synthetic construct
  source 1..27: mol_type = other DNA
ccagatctgagcctgggagctctctgg

SEQ ID NO: 53 - DNA, 12 bp, synthetic construct
  source 1..12: mol_type = other DNA
gttttagagcta

SEQ ID NO: 54 - DNA, 12 bp, synthetic construct
  source 1..12: mol_type = other DNA
gtttaagagcta

SEQ ID NO: 55 - DNA, 12 bp, synthetic construct
  source 1..12: mol_type = other DNA
gtttaagagata

SEQ ID NO: 56 - DNA, 13 bp, synthetic construct
  source 1..13: mol_type = other DNA
gtttaagagataa

SEQ ID NO: 57 - DNA, 12 bp, synthetic construct
  source 1..12: mol_type = other DNA
gtttaagagtta

SEQ ID NO: 58 - DNA, 12 bp, synthetic construct
  source 1..12: mol_type = other DNA
gtttaagagttc

SEQ ID NO: 59 - DNA, 12 bp, synthetic construct
  source 1..12: mol_type = other DNA
gtttaagagcgc

SEQ ID NO: 60 - DNA, 13 bp, synthetic construct
  source 1..13: mol_type = other DNA
gtttaagagcgtc

SEQ ID NO: 61 - DNA, 14 bp, synthetic construct
  source 1..14: mol_type = other DNA
gtttaagagcgtac

SEQ ID NO: 62 - DNA, 15 bp, synthetic construct
  source 1..15: mol_type = other DNA
gtttaagagcgtagc

SEQ ID NO: 63 - DNA, 16 bp, synthetic construct
  source 1..16: mol_type = other DNA
gtttaagagcgtagcg

SEQ ID NO: 64 - DNA, 17 bp, synthetic construct
  source 1..17: mol_type = other DNA
gtttaagagcgtagctg

SEQ ID NO: 65 - DNA, 18 bp, synthetic construct
  source 1..18: mol_type = other DNA
gtttaagagcgtcagctg

SEQ ID NO: 66 - DNA, 60 bp, synthetic construct
  source 1..60: mol_type = other DNA
gcgcaagtttaaataaggctagtccgttatcaacttgaaaaagtgggaccgagtcggtcc

SEQ ID NO: 67 - DNA, 14 bp, synthetic construct
  source 1..14: mol_type = other DNA
tagcaagtttaaat

SEQ ID NO: 68 - DNA, 14 bp, synthetic construct
  source 1..14: mol_type = other DNA
tatcaagtttaaat

SEQ ID NO: 69 - DNA, 15 bp, synthetic construct
  source 1..15: mol_type = other DNA
ttatcaagtttaaat

SEQ ID NO: 70 - DNA, 14 bp, synthetic construct
  source 1..14: mol_type = other DNA
taacaagtttaaat

SEQ ID NO: 71 - DNA, 14 bp, synthetic construct
  source 1..14: mol_type = other DNA
gaacaagtttaaat

SEQ ID NO: 72 - DNA, 14 bp, synthetic construct
  source 1..14: mol_type = other DNA
gcgcaagtttaaat

SEQ ID NO: 73 - DNA, 15 bp, synthetic construct
  source 1..15: mol_type = other DNA
gacgcaagtttaaat

SEQ ID NO: 74 - DNA, 16 bp, synthetic construct
  source 1..16: mol_type = other DNA
gtacgcaagtttaaat

SEQ ID NO: 75 - DNA, 17 bp, synthetic construct
  source 1..17: mol_type = other DNA
gctacgcaagtttaaat

SEQ ID NO: 76 - DNA, 18 bp, synthetic construct
  source 1..18: mol_type = other DNA
cgctacgcaagtttaaat

SEQ ID NO: 77 - DNA, 19 bp, synthetic construct
  source 1..19: mol_type = other DNA
cagctacgcaagtttaaat

SEQ ID NO: 78 - DNA, 20 bp, synthetic construct
  source 1..20: mol_type = other DNA
cagctgacgcaagtttaaat

SEQ ID NO: 79 - DNA, 46 bp, synthetic construct
  source 1..46: mol_type = other DNA
aaggctagtccgttatcaacttgaaaaagtgggaccgagtcggtcc

SEQ ID NO: 80 - DNA, 26 bp, synthetic construct
  source 1..26: mol_type = other DNA
tctgccatcaaagcgtgctcagtctg

SEQ ID NO: 81 - DNA, 42 bp, synthetic construct
  source 1..42: mol_type = other DNA
ttgacgcggttctatctagttacgcgttaaaccaactagaaa

SEQ ID NO: 82 - DNA, 101 bp, synthetic construct
  source 1..101: mol_type = other DNA
ggcccagactgagcacgtgagtttaagagcgcttctccacatgaggatcacccatgtggttgacgcggttctatctagttacgcgttaaaccaactagaaa

SEQ ID NO: 83 - DNA, 161 bp, synthetic construct
  source 1..161: mol_type = other DNA
ggatagggccctgaagaagggccctatctcttcgcgcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 84 - DNA, 101 bp, synthetic construct
  source 1..101: mol_type = other DNA
ggcccagactgagcacgtgagtttaagagctattctccacatgaggatcacccatgtggttgacgcggttctatctagttacgcgttaaaccaactagaaa

SEQ ID NO: 85 - DNA, 101 bp, synthetic construct
  source 1..101: mol_type = other DNA
ggcccagactgagcacgtgagtttaagagatattctccacatgaggatcacccatgtggttgacgcggttctatctagttacgcgttaaaccaactagaaa

SEQ ID NO: 86 - DNA, 102 bp, synthetic construct
  source 1..102: mol_type = other DNA
ggcccagactgagcacgtgagtttaagagataattctccacatgaggatcacccatgtggttgacgcggttctatctagttacgcgttaaaccaactagaaa

SEQ ID NO: 87 - DNA, 101 bp, synthetic construct
  source 1..101: mol_type = other DNA
ggcccagactgagcacgtgagtttaagagttattctccacatgaggatcacccatgtggttgacgcggttctatctagttacgcgttaaaccaactagaaa

SEQ ID NO: 88 - DNA, 101 bp, synthetic construct
  source 1..101: mol_type = other DNA
ggcccagactgagcacgtgagtttaagagttcttctccacatgaggatcacccatgtggttgacgcggttctatctagttacgcgttaaaccaactagaaa

SEQ ID NO: 89 - DNA, 101 bp, synthetic construct; identical to SEQ ID NO: 82
  source 1..101: mol_type = other DNA

SEQ ID NO: 90 - DNA, 102 bp, synthetic construct
  source 1..102: mol_type = other DNA
ggcccagactgagcacgtgagtttaagagcgtcttctccacatgaggatcacccatgtggttgacgcggttctatctagttacgcgttaaaccaactagaaa

SEQ ID NO: 91 - DNA, 103 bp, synthetic construct
  source 1..103: mol_type = other DNA
ggcccagactgagcacgtgagtttaagagcgtacttctccacatgaggatcacccatgtggttgacgcggttctatctagttacgcgttaaaccaactagaaa

SEQ ID NO: 92 - DNA, 104 bp, synthetic construct
  source 1..104: mol_type = other DNA
ggcccagactgagcacgtgagtttaagagcgtagcttctccacatgaggatcacccatgtggttgacgcggttctatctagttacgcgttaaaccaactagaaa

SEQ ID NO: 93 - DNA, 105 bp, synthetic construct
  source 1..105: mol_type = other DNA
ggcccagactgagcacgtgagtttaagagcgtagcgttctccacatgaggatcacccatgtggttgacgcggttctatctagttacgcgttaaaccaactagaaa

SEQ ID NO: 94 - DNA, 106 bp, synthetic construct
  source 1..106: mol_type = other DNA
ggcccagactgagcacgtgagtttaagagcgtagctgttctccacatgaggatcacccatgtggttgacgcggttctatctagttacgcgttaaaccaactagaaa

SEQ ID NO: 95 - DNA, 107 bp, synthetic construct
  source 1..107: mol_type = other DNA
ggcccagactgagcacgtgagtttaagagcgtcagctgttctccacatgaggatcacccatgtggttgacgcggttctatctagttacgcgttaaaccaactagaaa

SEQ ID NO: 96 - DNA, 161 bp, synthetic construct
  source 1..161: mol_type = other DNA
ggatagggccctgaagaagggccctatctcttctagcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 97 - DNA, 161 bp, synthetic construct
  source 1..161: mol_type = other DNA
ggatagggccctgaagaagggccctatctcttctatcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 98 - DNA, 162 bp, synthetic construct
  source 1..162: mol_type = other DNA
ggatagggccctgaagaagggccctatctcttcttatcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 99 - DNA, 161 bp, synthetic construct
  source 1..161: mol_type = other DNA
ggatagggccctgaagaagggccctatctcttctaacaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 100 - DNA, 161 bp, synthetic construct
  source 1..161: mol_type = other DNA
ggatagggccctgaagaagggccctatctcttcgaacaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 101 - DNA, 161 bp, synthetic construct; identical to SEQ ID NO: 83
  source 1..161: mol_type = other DNA

SEQ ID NO: 102 - DNA, 162 bp, synthetic construct
  source 1..162: mol_type = other DNA
ggatagggccctgaagaagggccctatctcttcgacgcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 103 - DNA, 163 bp, synthetic construct
  source 1..163: mol_type = other DNA
ggatagggccctgaagaagggccctatctcttcgtacgcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 104 - DNA, 164 bp, synthetic construct
  source 1..164: mol_type = other DNA
ggatagggccctgaagaagggccctatctcttcgctacgcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 105 - DNA, 165 bp, synthetic construct
  source 1..165: mol_type = other DNA
ggatagggccctgaagaagggccctatctcttccgctacgcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 106 - DNA, 166 bp, synthetic construct
  source 1..166: mol_type = other DNA
ggatagggccctgaagaagggccctatctcttccagctacgcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 107 - DNA, 167 bp, synthetic construct
  source 1..167: mol_type = other DNA
ggatagggccctgaagaagggccctatctcttccagctgacgcaagtttaaataaggctagtccgttatcaacttgaaaaagtgggaccgagtcggtcctctgccatcaaagcgtgctcagtctgttgacgcggttctatctagttacgcgttaaaccaactagaaa

SEQ ID NO: 108 - protein, 22 aa, synthetic construct
MDAQTRRRERRAEKQAQWKAAN

SEQ ID NO: 109 - protein, 129 aa, synthetic construct
ASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGLY

SEQ ID NO: 110 - protein, 93 aa, synthetic construct [sequence omitted]

SEQ ID NO: 111 - protein, 108 aa, synthetic construct
MGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 112 - protein, 218 aa, synthetic construct
EFMSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEKGT

SEQ ID NO: 113 - protein, 17 aa, synthetic construct
MRDHMVLHESVNAAGIT

SEQ ID NO: 114 - protein, 247 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSS

SEQ ID NO: 115 - protein, 19 aa, synthetic construct
EELLSKNYHLENEVARLKK

SEQ ID NO: 116 - protein, 134 aa, synthetic construct
SGEVQLQESGGGLVQPGGSLRLSCTASGVTISALNAMAMGWYRQAPGERRVMVAAVSERGNAMYRESVQGRFTVTRDFTNKMVSLQMDNLKPEDTAVYYCHVLEDRVDSFHDYWGQGTQVTVSSGAPGFSSISA

SEQ ID NO: 117 - protein, 15 aa, synthetic construct
PSRLEEELRRRLTEP

SEQ ID NO: 118 - protein, 180 aa, synthetic construct
GAPTQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 119 - protein, 300 aa, synthetic construct
TRVPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 120 - RNA, 22 nt, synthetic construct
  source 1..22: mol_type = other RNA
gttttagagctatgctgttttg

SEQ ID NO: 121 - RNA, 24 nt, synthetic construct
  source 1..24: mol_type = other RNA
taaaattgaacgatacgacaaaac

SEQ ID NO: 122 - RNA, 14 nt, synthetic construct
  source 1..14: mol_type = other RNA
taaaattgaacgat

SEQ ID NO: 123 - RNA, 11 nt, synthetic construct
  source 1..11: mol_type = other RNA
taaaattgaac

SEQ ID NO: 124 - RNA, 24 nt, synthetic construct
  source 1..24: mol_type = other RNA
gttttagagctagaacacacctgg

SEQ ID NO: 125 - RNA, 26 nt, synthetic construct
  source 1..26: mol_type = other RNA
taaaattgaacgatcttgtgtggacc

SEQ ID NO: 126 - RNA, 20 nt, synthetic construct
  source 1..20: mol_type = other RNA
gttttagagctatgctgccg

SEQ ID NO: 127 - RNA, 22 nt, synthetic construct
  source 1..22: mol_type = other RNA
taaaattgaacgatacgacggc

SEQ ID NO: 128 - RNA, 20 nt, synthetic construct
  source 1..20: mol_type = other RNA
gtttaagagctatgctgccg

SEQ ID NO: 129 - RNA, 22 nt, synthetic construct
  source 1..22: mol_type = other RNA
taaatttgaacgatacgacggc

SEQ ID NO: 130 - protein, 18 aa, synthetic construct
KRTADGSEFESPKKKRKV

SEQ ID NO: 131 - protein, 9 aa, synthetic construct
PAAKRVKLD

SEQ ID NO: 132 - protein, 16 aa, synthetic construct
KRPAATKKAGQAKKKK

SEQ ID NO: 133 - protein, 234 aa, synthetic construct
MEQKLISEEDLKRPAATKKAGQAKKKKGGSASGGSMDAQTRRRERRAEKQAQWKAANSGGSSGGSKRTADGSEFESPKKKRKVSGGSSGGSGGPAAKRVKLDGSGASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGLY

(19) United States
(12) Patent Application Publication
(10) Pub. No.: US 2026/0176627 A1
(43) Pub. Date: Jun. 25, 2026

(54) DUAL-RNA-GUIDED, SPLIT-PEGRNA RECORDER FOR MOLECULAR INTERACTION

(71) Applicant: University of Washington, Seattle, WA (US)

(72) Inventors: Junhong CHOI, Seattle, WA (US); Jay SHENDURE, Seattle, WA (US); Wei CHEN, Seattle, WA (US)

(21) Appl. No.: 19/127,527

(22) PCT Filed: Nov. 16, 2023
(86) PCT No.: PCT/US2023/079962

§ 371 Date: May 6, 2025

Related U.S. Application Data
(60) Provisional application No. 63/384,259, filed on Nov. 18, 2022.

(51) Int. Cl. C12N 15/113; A61K 48/00; C12Q 1/6869

(52) U.S. Cl. CPC C12N 15/113; A61K 48/00; C12Q 1/6869; C12N 2310/321

(57) ABSTRACT

Split-pegRNA recorder reagents and methods are provided that provide specific molecular interactions within cells induce precise genome editing that inserts an event-specific barcode sequence into the predetermined genomic locus of DNA Tape. The split pegRNAs include (a) a crRNA component, including in 5′ to 3′ order (i) a spacer sequence for a genomic locus of interest; (ii) a crRNA repeat sequence necessary for binding to a CRISPR-Cas protein; and (iii) a first RNA extension; and (b) a petracrRNA component, including in 5′ to 3′ order (i) a second RNA extension; (ii) a petracrRNA antirepeat sequence necessary for binding to a CRISPR-Cas protein; (iii) a gRNA scaffold; wherein the crRNA component and the petracrRNA component are not covalently bound to each other.

### CROSS REFERENCE

This application claims priority to U.S. Provisional Patent Application Ser. No. 63/384,259 filed Nov. 18, 2022, incorporated by reference herein in its entirety.

### FEDERAL FUNDING STATEMENT

This invention was made with government support under Grant Nos. HG010632 and HG011586, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Nov. 15, 2023 having the file name “22-1918-WO.xml” and is 139,040 bytes in size.

### BACKGROUND

Molecular recording is a rapidly maturing method that records biological events to the in vivo storage media for later reconstruction. DNA-based memory devices achieve molecular recording by converting specific biological events to altered genomic sequences. Previously, we and others have demonstrated molecular recording devices based on prime editing, recording cell lineage and transcription activation events via precise genome editing. Here the edits are the insertion of an event-specific barcode sequence to a target (“DNA Tape”), where the order of events is encoded within the physical location of insertion barcodes within the target. In recording cell lineage information, multiple constitutively transcribed prime editing guide RNAs (pegRNAs) were used to stochastically edit DNA Tape, resulting in an accumulation of edits similar to a natural mutation across the genome for inferring lineage relationships across different organisms. In recording transcription activation events, DNA transcription enhancer elements are used as a sensor of cellular events to drive transcription of specific pegRNAs, recording the identity of cellular events to DNA Tape.

In addition to recording the transcription activation events within living cells, physical interactions between two biomolecules are another important subset of cellular events for possible recording. Physical interactions between two biomolecules are underpinning complex biological processes, largely determining the efficiency and error rate of each biochemical reaction. Quantifying physical interactions between two molecules in their native environment is challenging, where accurate measurements are often done with in vitro systems containing purified components.

### SUMMARY

In one aspect, the disclosure provides split pegRNA comprising:
(a) a crRNA component, comprising in 5′ to 3′ order:
   (i) a spacer sequence for a genomic locus of interest;
   (ii) a crRNA repeat sequence necessary for binding to a CRISPR-Cas protein; and
   (iii) a first RNA extension; and
(b) a petracrRNA component, comprising in 5′ to 3′ order:
   (i) a second RNA extension;
   (ii) a petracrRNA antirepeat sequence necessary for binding to a CRISPR-Cas protein;
   (iii) a gRNA scaffold;
   (iv) an optional reverse transcriptase template sequence (RTS); and
   (v) an optional primer binding site (PBS);
wherein the crRNA component and the petracrRNA component are not covalently bound to each other;
wherein:
   (I) the first RNA extension is capable of binding to a first protein, and the second RNA extension is capable of binding to a second protein, wherein the first protein and the second protein are capable of binding to each other; or
   (II) wherein one domain of the first protein and the first RNA extension are capable of binding to each other, and another domain of the first protein and the second RNA extension are capable of binding to each other; or
   (III) the first RNA extension is capable of binding to a first protein, and the second RNA extension is capable of binding to a second protein, wherein there exists a chemical that induce the binding between the first protein and the second protein when introduced to cells or naturally synthesized within cells.

In one embodiment, the CRISPR-Cas protein is selected from the group consisting of Cas9, nickases, nucleases, deactivated Cas9, modified Cas9 in Base Editors, and CRISPR-Cas proteins used with other epigenetic effector modules, e.g. CRISPRa/i. In another embodiment, one of the first and second RNA extensions comprises an MS2 RNA stem loop, BoxB RNA stem loop, PP7 RNA stem loop, or HIV-1 TAR stem loop, and the other comprises any other RNA stem loop with a known stable protein binding partner.

In some embodiments, one domain of the first protein and the first RNA extension are capable of binding to each other, and another domain of the first protein and the second RNA extension are capable of binding to each other, either constitutively or dynamically controlled by additional chemicals that induce binding. In one such embodiment, the first protein comprises MCP domain for binding MS2 RNA hairpin, LambdaN domain for binding BoxB RNA hairpin, PCP domain for binding PP7 RNA hairpin, or HIV-1 Tat domain for binding TAR RNA hairpin.

In another embodiment, the first RNA extension is capable of binding to a first protein, and the second RNA extension is capable of binding to a second protein, wherein there exists a chemical that induces the binding between the first protein and the second protein when introduced to cells or naturally synthesized within cells. In some embodiments, the chemical is a small molecule; in other embodiments, the small molecule is selected from rapamycin and abscisic acid. In further embodiments, the small molecule is rapamycin, and wherein the first protein and the second protein comprise FKBP (FK506 Binding Proteins) and FRB (FKBP-Rapamycin Binding). In other embodiments, the small molecule is abscisic acid (ABA)- and the first protein and the second protein comprise pyrabactin resistance domain (PYL) and ABA-insensitive (ABI) domain.

The disclosure also provides nucleic acids encoding the crRNA or petracrRNA of any embodiment herein, expression vectors comprising the nucleic acid operatively linked to a suitable control element, such as a promoter; host cells comprising the split pegRNA, nucleic acid, and/or expression vector of any embodiment, and kits comprising the split-pegRNA of any preceding claim.

The disclosure also provides methods for recording a protein-protein and/or protein-RNA interaction within a cell, comprising expressing in the cell:
(a) the split pegRNA of any embodiment,
(b) the first and, if necessary, the second protein as recited herein, and
(c) the modified or unmodified CRISPR-Cas protein of any embodiment;
wherein dimerization of the first and second RNA extensions of the split pegRNA via binding to the first and, if necessary, the second protein in the cell induces formation of functioning guide RNA for genome or epigenome editing via the CRISPR-Cas protein, thereby inducing genome or epigenome editing to edit genomic DNA in the cell and recording of the protein-protein and/or protein-RNA interaction into genomic DNA in the cell.

In another embodiment the disclosure provides methods for recording a protein-protein and/or protein-RNA interaction within a cell, comprising expressing in the cell:
(a) the split pegRNA of any embodiment,
(b) the first and, if necessary, the second protein of any embodiment, and
(c) the modified or unmodified CRISPR-Cas protein of any embodiment;
wherein dimerization of the first and second RNA extensions of the split pegRNA via binding to the first and the second protein in the cell, which is controlled by chemicals that induce binding between the first and second proteins, induces formation of functioning guide RNA for genome or epigenome editing via the CRISPR-Cas protein, thereby controlling genome or epigenome editing to edit genomic DNA in the cell with chemicals that control protein-protein dimerization.

### DESCRIPTION OF THE FIGURES

FIG. 1. Testing editing efficiencies of split-pegRNA. a. We have tested three classes for split-pegRNA designs: (top) Cas9-binding scaffold is split at the repeat-antirepeat junction in crRNA-tracrRNA complex, which is joined through a GAAA RNA tetraloop in the sgRNA. (center) pegRNA is split to sgRNA and trans-peRNA. (bottom) Self-splicing ribozyme is inserted into pegRNA, which can be split into two parts. b. A dimerization of crRNA and prime editing tracrRNA (petracrRNA) for Cas9 activity is guided by RNA annealing sequences that are reverse-complementary to each other. c. Different designs of annealing sequences (from top to bottom: SEQ ID NOs: 120, 121, 7, 122, 123, 124, 125, 126, 127, 128, and 129). d-e. Editing efficiencies for prime editing (CTT insertion to HEK3 native genomic locus) using matching (d) and mixed (e) crRNA/petracrRNA pairs. CTT insertion efficiency using epegRNA was used for the positive control in the assay.

FIG. 2. Recording heterodimerization of split-GFP molecules. a. The schematic of prime editing induced by dimerization of split-GFP. MS2-MCP and BoxB-λN RNA-protein interactions serve as adaptors to transduce protein dimerization signals to crRNA-petracrRNA dimerization for forming functional pegRNA. b. Editing efficiency measured with or without split-GFP to induce crRNA-petracrRNA dimerization. c. Testing crRNA-petracrRNA pairs with different RNA-RNA interaction strengths with split-GFP dimerization systems. The upper four base pairs of the Cas9-binding region of repeat: anti-repeat duplex were altered to generate 12 pairs of crRNA-MS2/BoxB-petracrRNA designs. The editing efficiency is normalized with the eCTT positive control included in each experiment (targeting HEK3 locus with CTT insertion at position +0 using standard epegRNA), to control for variable transfection efficiencies. Two normalized editing efficiencies were measured for each pair of RNAs: one with tagged split-GFP to promote dual-RNA-guide formation, and one with standard, untagged GFP to measure the background editing level non-specific to protein-protein proximity.

FIG. 3. Recording protein-protein interaction and small molecule exposure using Split-pegRNA recorders. a-b. Design of Split-pegRNA recorder with protein adaptors to record protein-protein interaction (a) and exposure to small molecule (b). c. Normalized editing efficiency of different pairs of constructs tagged with MCP and LambdaN adaptors. Editing efficiencies are scaled (“normalized”) to positive editing control of CTT insertion to HEK3 locus by epegRNA (“eCTT control”). In MCP-LambdaN condition, a single protein-expression construct with MCP tethered with LambdaN was transfected instead of a pair of protein-expression constructs. In FKBP (FK506 Binding Proteins) and FRB (FKBP-Rapamycin Binding) conditions, different concentrations of Rapamycin were added to the cell culture to induce dimerization of FKBP-MCP and LambdaN-FRB. In ABI and PYL conditions, different concentrations of abscisic acid (ABA) was added to the cell culture to induce dimerization of PYL-MCP and LambdaN-ABI.

### DETAILED DESCRIPTION

As used herein, the singular forms “a”, “an” and “the” include plural referents unless the context clearly dictates otherwise.

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Disclosed herein are reagents and methods that can be used, for example, in a strategy named “Split-pegRNA recorders”, where specific molecular interactions within cells induce precise genome editing that inserts an event-specific barcode sequence into the predetermined genomic locus of DNA Tape. The molecular interaction between two protein or RNA molecules is sensed by two adaptor modules attached to dual-RNA-guide molecules. The dimerization of adaptor modules induces the formation of functioning guide RNA for prime editing, inducing prime editing to record its occurrence onto the stable genomic DNA medium. As disclosed in the examples, the inventors demonstrate that genome editing efficiency depends on the strength of dimerization interactions, therefore faithfully measuring the interaction strength between two molecules.

In one aspect, the disclosure provides split pegRNAs. To record the physical interaction between two molecules within the living cell, the inventors designed a precision genome editing system based on prime editing, where the prime editing guide RNA (pegRNA) is split into two complementary parts that are functional only if they form a stable heterodimer. The pegRNA is split within the sgRNA scaffold, such that tracrRNA is extended with the prime editing-specific reverse-transcription template, referred to as “petracrRNA” (prime editing trans-activating CRISPR RNA). The pegRNAs are split at the sgRNA scaffold repeat-antirepeat junction, forming crRNA and petracrRNA molecules as defined here. Here, the petracrRNA carries the 3′-end extension necessary for the prime editing (primer binding sites and RT-template sequences for generating the 3′ overhang on the nicked genomic DNA). The upper stem-loop of the repeat-antirepeat region of gRNA is not necessary for Cas9 function and is replaced it with the two RNA extensions that are reverse-complementary to each other.

Thus, in one embodiment the disclosure provides split pegRNAs comprising:
(a) a crRNA component, comprising in 5′ to 3′ order:
   (i) a spacer sequence for a genomic locus of interest;
   (ii) a crRNA repeat sequence necessary for binding to a CRISPR-Cas protein; and
   (iii) a first RNA extension; and
(b) a petracrRNA component, comprising in 5′ to 3′ order:
   (i) a second RNA extension;
   (ii) a petracrRNA antirepeat sequence necessary for binding to a CRISPR-Cas protein;
   (iii) a gRNA scaffold;
   (iv) an optional reverse transcriptase template sequence (RTS) that is specific for a specific genomic locus of interest; and
   (v) an optional primer binding site (PBS) that is specific for a specific genomic locus of interest;
wherein the crRNA component and the petracrRNA component are not covalently bound to each other;
wherein:
   (I) the first RNA extension is capable of binding to a first protein, and the second RNA extension is capable of binding to a second protein, wherein the first protein and the second protein are capable of binding to each other; or
   (II) wherein one domain of the first protein and the first RNA extension are capable of binding to each other, and another domain of the first protein and the second RNA extension are capable of binding to each other.

The spacer sequence may be any sequence as appropriate for an intended genomic locus to be targeted. The spacer sequence is identical to the genomic locus of interest on the strand where PAM (protospacer adjacent motif) is selected. The canonical PAM is the sequence 5′-NGG-3′ (“NGG”), where “N” is any nucleotide followed by two guanine (“G”) nucleotides. The spacer sequence in Cas9-based prime editing is adjacent to an NGG PAM sequence motif. The length of the spacer sequence may be about 20-bp, but shorter or longer can be used at a lower efficiency (i.e., 5-50 bp; 10-40 bp; 15-35 bp; 10-30 bb; 15-35 bp; etc.) It will be clear to those of skill in the art what spacer sequence can be used in view of a genomic locus of interest.

As used herein, the CRISPR-Cas protein is any CRISPR-Cas protein based on dual-RNA-guide system, including but not limited to Cas9, nickases, nucleases, deactivated Cas9, used with other epigenetic effector modules, e.g. CRISPRa/i, and modified Cas9 such as Base Editors. In some embodiments, CRISPR-Cas protein is a reverse-transcriptase tethered Cas9 nickase for genome editing. In other embodiments, other systems such as Cas9 nuclease or deactivated Cas9 tethered with other transcription activator (e.g., VP64 or VPR) or inhibitor (e.g., KRAB) domains can be used with split-pegRNA. In the examples provided herein, Cas9 is used an exemplary CRISPR-Cas protein.

In some embodiments, the CRISPR-Cas protein can be composed of a Cas polypeptide having nickase activity, a reverse transcriptase, and a guide molecule. The Cas polypeptide can lack nuclease activity. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

In one embodiment, the CRISPR-Cas protein is selected from the group consisting of Cas9, nickases, nucleases, deactivated Cas9, modified Cas9 in Base Editors, and CRISPR-Cas proteins used with other epigenetic effector modules, e.g. CRISPRa/i. In one embodiment, the CRISPR-Cas protein comprises Cas9.

In a further embodiment, the first RNA extension is capable of binding to a first protein, and the second RNA extension is capable of binding to a second protein, wherein the first protein and the second protein are capable of binding to each other. In this embodiment, the first protein and the second protein may be any interacting proteins that RNA extensions can be designed to bind to. Similarly, the first RNA extension and the second RNA extension may comprise any nucleotide sequence and/or secondary structure that can bind to appropriate interacting first and second proteins, respectively. In some embodiments, one or both RNA extensions comprise an RNA stem loop structure. In various embodiments, one of the first and second RNA extensions comprises an MS2 RNA stem loop, BoxB RNA stem loop, PP7 RNA stem loop, or HIV-1 TAR stem loop, and the other comprises any other RNA stem loop with a known stable protein binding partner.

In a specific embodiment, one of the first and second RNA extensions comprises an MS2 RNA stem loop, and the other comprises a BoxB RNA stem loop. In this embodiment, the MS2 RNA stem loop is capable of binding to MS2 coat protein (MCP) and the BoxB RNA stem loop is capable of binding to LambdaN protein, with MCP and LambdaN protein being capable of interacting.

LambdaN amino-acid sequence
(SEQ ID NO: 108)
MDAQTRRRERRAEKQAQWKAAN
MCP amino-acid sequence
(SEQ ID NO: 109)
ASNFTQFVLVDNGGTGDVTVAPSNFANGVAEWISSNSRSQAYKVTCSVR
QSSAQKRKYTIKVEVPKVATQTVGGVELPVAAWRSYLNMELTIPIFATN
SDCELIVKAMQGLLKDGNPIPSAIAANSGLY

In another embodiment, one of the first and second RNA extensions comprises a PP7 RNA stem loop, and the other comprises an HIV-1 TAR stem loop. In this embodiment, the PP7 RNA stem loop is capable of binding to the coat protein of bacteriophage PP7 and the HIV-1 TAR stem loop is capable of binding to the HIV Tat protein.

It will be understood by those of skill in the art that these first and second RNA extensions are exemplary, and that any suitable RNA extensions can be used that are capable of binding to interacting first and second proteins of interest. It will further be understood by those of skill in the art, that the first and second proteins may be functionalized so that their interaction produces a specific result. In some non-limiting embodiments, the first and second proteins may each be fused a first member of a split reporter protein, including but not limited to a split green fluorescent protein, as exemplified in the examples below. In this embodiment, binding of the first and second RNA extensions to the first and second protein may result in reconstitution of the reporter protein signal (exemplified by GFP), permitting visualization of the interaction. In another non-limiting embodiment, the first and second proteins are functionalized by fusing to chemically inducible dimerization domains such as binding to the FKBP and FRB protein domains of the mTOR pathway, which dimerize only in the presence of the chemical rapamycin.

FRB amino-acid sequence
(SEQ ID NO: 110)
ILWHEMWHEGLEEASRLYFGERNVKGMFEVLEPLHAMMERGPQTLKETS
FNQAYGRDLMEAQEWCRKYMKSGNVKDLTQAWDLYYHVFRRISK
FKBP amino-acid sequence
(SEQ ID NO: 111)
MGVQVETISPGDGRTFPKRGQTCVVHYTGMLEDGKKFDSSRDRNKPFKF
MLGKQEVIRGWEEGVAQMSVGQRAKLTISPDYAYGATGHPGIIPPHATL
VFDVELLKLE

In this embodiment, exemplified in the examples, exposure of cells to a small molecule or chemical such as rapamycin can be recorded into the genome using specifically engineered split-pegRNA recorders according to the disclosure. This, in turn, suggests that the genome editing activities can be controlled by the chemical dose introduced to cells, where different genome editing outcomes can be controlled by different chemicals in parallel. As will be understood by those of skill in the art based on the teachings herein, these are exemplary embodiments only, and the first and second proteins may be functionalized in any way as appropriate for an intended use.

In these various embodiments, a nucleotide sequence of the MS2, BoxB, PP7, and HIV-TAR stem loop structures may vary widely, as will be understood by those of skill in the art. In various non-limiting embodiments, the MS2 RNA stem loop comprises or consists of the nucleic acid sequence selected from the group consisting of SEQ ID NO: 1-2, and the BoxB RNA stem loop comprises or consists of the nucleic acid sequence selected from the group consisting of SEQ ID NO:3-4; wherein optional nucleotides (in parentheses) may be present or may be deleted.

### MS2 RNA Stem Loop

NRNDSASSANCAS′S′S′N′N′Y′N′ (SEQ ID NO: 1), wherein the variable positions are written following the IUPAC ambiguity nucleotide code. The key for the IUPAC ambiguity nucleotide code is as follows:

$$R = A or G ;$$

$$K = G or T / U ;$$

$$S or {S}^{′} = G or C ;$$

$${Y}^{′} = C or T / U$$

$$M = A or C ;$$

$$W = A or T / U ;$$

$$B = not A ( C , G or T / U ) ;$$

$$H = not G ( A , C or T / U ) ;$$

$$N or {N}^{′} = any nucleotide ;$$

$$D = not C ( A , G or T / U ) ; and$$

$$V = not T / U ( A , C or G ) .$$

(SEQ ID NO: 2)
UUCUCCACAUGAGGAUCACCCAUGUGG
BoxB RNA stem loop
(SEQ ID NO: 3)
GGGCCCUGAAGAAGGGCCC
(SEQ ID NO: 4)
GGAUAGGGCCCUGAAGAAGGGCCCUAUCUCUUC

In one embodiment, the PP7 RNA stem loop comprises or consists of the nucleic acid sequence: GGAGCAGACGAUAUGGCGUCGCUCC (SEQ ID NO:5), and the HIV-1 TAR stem loop comprises or consists of the nucleic acid sequence of SEQ ID NO:6.

(SEQ ID NO: 6)
CCAGAUCUGAGCCUGGGAGCUCUCUGG.

In another embodiment, the first RNA extension and the second RNA extension bind to different domains of the same protein. In this embodiment, one domain of the first protein and the first RNA extension are capable of binding to each other, and another domain of the first protein and the second RNA extension are capable of binding to each other. This embodiment can be used, for example, to determine whether a protein is made in the cell or not, and the complete split-pegRNA is only made when the protein is made to bring the crRNA and petracrRNA together.

In other embodiments, the first protein comprises MCP domain for binding MS2 RNA hairpin, LambdaN domain for binding BoxB RNA hairpin, PCP domain for binding PP7 RNA hairpin, or HIV-1 Tat domain for binding TAR RNA hairpin.

The crRNA repeat sequence necessary for binding to a CRISPR-Cas protein and the petracrRNA antirepeat sequence necessary for binding to a given CRISPR-Cas protein are known by those of skill in the art. These sequences are needed for gRNA to bind CRISPR-Cas protein. Non-limiting exemplary embodiments of crRNA repeat sequence necessary for binding to a CRISPR-Cas protein comprise or consist of nucleotide sequences found in Table 1 in the column entitled “crRNA_repeat”. Non-limiting exemplary embodiments of petracrRNA antirepeat sequences necessary for binding to a CRISPR-Cas protein comprise or consist of nucleotide sequences found in Table 2 in the column entitled “petracrRNA_antirepeat”.

In some embodiments, the crRNA repeat comprises or consists of the nucleic acid sequence selected from the group consisting of SEQ ID NO:7-19.

(SEQ ID NO: 7)
GUUUUAGAGCUA
(SEQ ID NO: 8)
GUUUAAGAGCUA
(SEQ ID NO: 9)
GUUUAAGAGAUA
(SEQ ID NO: 10)
GUUUAAGAGAUAA
(SEQ ID NO: 11)
GUUUAAGAGUUA
(SEQ ID NO: 12)
GUUUAAGAGUUC
(SEQ ID NO: 13)
GUUUAAGAGCGC
(SEQ ID NO: 14)
GUUUAAGAGCGUC
(SEQ ID NO: 15)
GUUUAAGAGCGUAC
(SEQ ID NO: 16)
GUUUAAGAGCGUAGC
(SEQ ID NO: 17)
GUUUAAGAGCGUAGCG
(SEQ ID NO: 18)
GUUUAAGAGCGUAGCUG
(SEQ ID NO: 19)
GUUUAAGAGCGUCAGCUG.

In another embodiment, the petracrRNA scaffold comprises or consists of the nucleic acid sequence selected from the group consisting of SEQ ID NO:20-32.

(SEQ ID NO: 20)
GCGCAAGUUUAAAUAAGGCUAGUCCGUUAUCAACUUGAAAAAGUGGGAC
CGAGUCGGUCC;
(SEQ ID NO: 21)
UAGCAAGUUUAAAU
(SEQ ID NO: 22)
UAUCAAGUUUAAAU
(SEQ ID NO: 23)
UUAUCAAGUUUAAAU
(SEQ ID NO: 24)
UAACAAGUUUAAAU
(SEQ ID NO: 25)
GAACAAGUUUAAAU
(SEQ ID NO: 26)
GCGCAAGUUUAAAU
(SEQ ID NO: 27)
GACGCAAGUUUAAAU
(SEQ ID NO: 28)
GUACGCAAGUUUAAAU
(SEQ ID NO: 29)
GCUACGCAAGUUUAAAU
(SEQ ID NO: 30)
CGCUACGCAAGUUUAAAU
(SEQ ID NO: 31)
CAGCUACGCAAGUUUAAAU
(SEQ ID NO: 32)
CAGCUGACGCAAGUUUAAAU

As will be understood by those of skill in the art, the gRNA scaffold may comprise any nucleotide sequence for functioning as a gRNA scaffold in the prime editing process, and gRNA scaffolds used in the examples that follow may comprise many nucleotide modifications while still functioning similarly. In one embodiment, the gRNA scaffold comprises or consists of a nucleic acid sequence at least 25%, 30%, 40%, 50%, 60%, 70%, 80%, 90%, 95%, 98%, or 100% identical to the nucleic acid sequence of SEQ ID NO:33.

(SEQ ID NO: 33)
AAGGCUAGUCCGUUAUCAACUUGAAAAAGUGGGACCGAGUCGGUCC.

The reverse transcriptase template sequence (RTS); and primer binding site (PBS) are optional. As will be understood by those of skill in the art, the RTS and PBS are specific for the genomic locus of interest of interest. The RTS includes the programmed genome editing outcome and is complementary to a nucleotide sequence downstream of the Cas9-generated nick on the genomic locus of interest, which act as a template for a reverse transcriptase enzyme. The PBS is complementary to a nucleotide sequence downstream of the Cas9-generated nick on the genomic locus of interest to permit binding of a reverse transcriptase. In other words, the spacer sequence determines where to target within the genome, the PBS sequence is complementary to the part of the spacer sequence for binding to the genomic DNA nicked with Cas9-nickase, and the RTS sequence includes an intended editing outcome as well as sequence complementary to the spacer sequence to enhance DNA repair. In one embodiment, the petracrRNA include the RTS and PBS that is specific for the genomic locus of interest. In these embodiments, and as will be understood by those of skill in the art, the nucleotide sequence of the RTS and PBS domains will vary depending on the type of edit to be made using the split-pegRNA. In other embodiments, the RTS and PBS are absent. For example, RTS and PBS sequence can be entirely omitted and used with the Cas9 nuclease or Cas9 modified to epigenetically inhibit or activate transcription (e.g., Cas9-KRAB or Cas9-VPR). In one non-limiting and exemplary embodiment, when the RTS and PBS are present, the RTS and PBS combination may comprise or consist of the nucleic acid sequence of SEQ ID NO: 34. UCUGCCAUCAAAGCGUGCUCAGUCUG (SEQ ID NO:34).

In another embodiment, the crRNA component and/or the petracrRNA component further comprises an RNA stabilization domain at their 3′ terminus. When present, any suitable RNA stabilization domain may be used, including an RNA pseudoknot, a RNA stem-loop, a Zikavirus exoribonuclease-resistant RNA motif, a G-quadruplex, or a stem-loop aptamer. In one embodiment, the RNA stabilization domain comprises or consists of the nucleic acid sequence of SEQ ID NO:35

(SEQ ID NO: 35)
UUGACGCGGUUCUAUCUAGUUACGCGUUAAACCAACUAGAAA.

In various embodiments, the crRNA component comprises the genus X1-X2, wherein X1 is a spacer sequence for a genomic locus of interest, and X2 comprises or consists of a nucleic acid sequence of the formula B1-B2-B3, wherein B1, B2, and B3, respectively, comprise or consist of, in 5′ to 3′ order:
SEQ ID NO:7-SEQ ID NO:2-SEQ ID NO:35;
SEQ ID NO:8-SEQ ID NO:2-SEQ ID NO:35;
SEQ ID NO:9-SEQ ID NO:2-SEQ ID NO:35;
SEQ ID NO:10-SEQ ID NO:2-SEQ ID NO:35;
SEQ ID NO: 11-SEQ ID NO:2-SEQ ID NO:35;
SEQ ID NO:12-SEQ ID NO:2-SEQ ID NO:35;
SEQ ID NO:13-SEQ ID NO:2-SEQ ID NO:35;
SEQ ID NO:14-SEQ ID NO:2-SEQ ID NO:35;
SEQ ID NO: 15-SEQ ID NO:2-SEQ ID NO:35;
SEQ ID NO: 16-SEQ ID NO:2-SEQ ID NO:35;
SEQ ID NO:17-SEQ ID NO:2-SEQ ID NO:35;
SEQ ID NO: 18-SEQ ID NO:2-SEQ ID NO:35; or
SEQ ID NO:19-SEQ ID NO:2-SEQ ID NO:35.

These embodiments are shown in Table 1.

TABLE 1
(5′ to 3′, crRNA repeat-MS2-RNA stabilization sequence)

| | crRNA_repeat | MS2 | RNA stabilization sequence |
|---|---|---|---|
| crRNA- | GUUUAAGAGCUA | UUCUCCACAUGAGGAUCACC | UUGACGCGGUUCUAUCUAGUUACGCGUU |
| MS2- | (SEQ ID | CAUGUGG (SEQ ID | AAACCAACUAGAAA (SEQ ID |
| GCUA | NO: 8) | NO: 2) | NO: 35) |
| crRNA- | GUUUAAGAGAUA | UUCUCCACAUGAGGAUCACC | UUGACGCGGUUCUAUCUAGUUACGCGUU |
| MS2- | (SEQ ID | CAUGUGG (SEQ ID | AAACCAACUAGAAA (SEQ ID |
| GAUA | NO: 9) | NO: 2) | NO: 35) |
| crRNA- | GUUUAAGAGAUA | UUCUCCACAUGAGGAUCACC | UUGACGCGGUUCUAUCUAGUUACGCGUU |
| MS2- | A (SEQ ID | CAUGUGG (SEQ ID | AAACCAACUAGAAA (SEQ ID |
| GAUAA | NO: 10) | NO: 2) | NO: 35) |
| crRNA- | GUUUAAGAGUUA | UUCUCCACAUGAGGAUCACC | UUGACGCGGUUCUAUCUAGUUACGCGUU |
| MS2- | (SEQ ID | CAUGUGG (SEQ ID | AAACCAACUAGAAA (SEQ ID |
| GUUA | NO: 11) | NO: 2) | NO: 35) |
| crRNA- | GUUUAAGAGUUC | UUCUCCACAUGAGGAUCACC | UUGACGCGGUUCUAUCUAGUUACGCGUU |
| MS2- | (SEQ ID | CAUGUGG (SEQ ID | AAACCAACUAGAAA (SEQ ID |
| GUUC | NO: 12) | NO: 2) | NO: 35) |
| crRNA- | GUUUAAGAGCGC | UUCUCCACAUGAGGAUCACC | UUGACGCGGUUCUAUCUAGUUACGCGUU |
| MS2- | (SEQ ID | CAUGUGG (SEQ ID | AAACCAACUAGAAA (SEQ ID |
| GCGC | NO: 13) | NO: 2) | NO: 35) |
| crRNA- | GUUUAAGAGCGU | UUCUCCACAUGAGGAUCACC | UUGACGCGGUUCUAUCUAGUUACGCGUU |
| MS2- | C (SEQ ID | CAUGUGG (SEQ ID | AAACCAACUAGAAA (SEQ ID |
| GCGUC | NO: 14) | NO: 2) | NO: 35) |
| crRNA- | GUUUAAGAGCGU | UUCUCCACAUGAGGAUCACC | UUGACGCGGUUCUAUCUAGUUACGCGUU |
| MS2- | AC (SEQ ID | CAUGUGG (SEQ ID | AAACCAACUAGAAA (SEQ ID |
| GCGUAC | NO: 15) | NO: 2) | NO: 35) |
| crRNA- | GUUUAAGAGCGU | UUCUCCACAUGAGGAUCACC | UUGACGCGGUUCUAUCUAGUUACGCGUU |
| MS2- | AGC (SEQ ID | CAUGUGG (SEQ ID | AAACCAACUAGAAA (SEQ ID |
| GCGUAG | NO: 16) | NO: 2) | NO: 35) |
| C | | | |
| crRNA- | GUUUAAGAGCGU | UUCUCCACAUGAGGAUCACC | UUGACGCGGUUCUAUCUAGUUACGCGUU |
| MS2- | AGCG ( SEQ ID | CAUGUGG (SEQ ID | AAACCAACUAGAAA (SEQ ID |
| GCGUAG | NO: 17) | NO: 2) | NO: 35) |
| CG | | | |
| CrRNA- | GUUUAAGAGCGU | UUCUCCACAUGAGGAUCACC | UUGACGCGGUUCUAUCUAGUUACGCGUU |
| MS2- | AGCUG (SEQ ID | CAUGUGG (SEQ ID | AAACCAACUAGAAA (SEQ ID |
| GCGUAG | NO: 18) | NO: 2) | NO: 35) |
| CUG | | | |
| crRNA- | GUUUAAGAGCGU | UUCUCCACAUGAGGAUCACC | UUGACGCGGUUCUAUCUAGUUACGCGUU |
| MS2- | CAGCUG (SEQ | CAUGUGG (SEQ ID | AAACCAACUAGAAA (SEQ ID |
| GCGUCA | ID NO: 19) | NO: 2) | NO: 35) |
| GCUG | | | |

In another embodiment, the crRNA component comprises the genus X1-X2, wherein X1 is a spacer sequence for a genomic locus of interest, and X2 comprises or consists of the nucleic acid sequence of SEQ ID NO:36.

(SEQ ID NO: 36)
GGCCCAGACUGAGCACGUGAGUUUAAGAGCGCUUCUCCACAUGAGGAUC
ACCCAUGUGGUUGACGCGGUUCUAUCUAGUUACGCGUUAAACCAACUAG
AAA

In a further embodiment, the petracrRNA component comprises a nucleic acid sequence of the genus Z1-Z2-Z3-Z4-Z5, wherein
Z1 comprises or consists of a BoxB nucleotide sequence selected from the group consisting of SEQ ID NO:3-4;
Z2 comprises or consists of a petracrRNA antirepeat nucleotide sequence selected from the group consisting of SEQ ID NO:20-32;
Z3 comprises a gRNA scaffold
Z4 comprises an RTT and PBS sequence, or is absent; and
Z5 comprises an RNA stabilization sequence or is absent.

In one such embodiment, Z5 is present, and comprises or consists of the RNA stabilization sequence of SEQ ID NO:35. In another embodiment, Z4 is present, and comprises or consists of a nucleic acid sequence specific for the genomic locus of interest. In another embodiment, Z3 comprises a nucleic acid sequence at least 25%, 30%, 40%, 50%, 60%, 70%, 80%, 90%, 95%, 98%, or 100% identical to the gRNA scaffold nucleotide sequence of SEQ ID NO:33.

In exemplary embodiments, the petracrRNA component comprises or consists of a nucleic acid sequence of the formula Jan. 2, 2003-04, wherein 01, 02, 03, and 04, respectively, comprise or consist of, in 5′ to 3′ order:
SEQ ID NO:4-SEQ ID NO:21-SEQ ID NO:33-SEQ ID NO:35;
SEQ ID NO:4-SEQ ID NO:22-SEQ ID NO:33-SEQ ID NO:35;
SEQ ID NO:4-SEQ ID NO:23-SEQ ID NO:33-SEQ ID NO:35;
SEQ ID NO:4-SEQ ID NO:24-SEQ ID NO:33-SEQ ID NO:35;
SEQ ID NO:4-SEQ ID NO:25-SEQ ID NO:33-SEQ ID NO:35;
SEQ ID NO:4-SEQ ID NO:26-SEQ ID NO:33-SEQ ID NO:35;
SEQ ID NO:4-SEQ ID NO:27-SEQ ID NO:33-SEQ ID NO:35;
SEQ ID NO:4-SEQ ID NO:28-SEQ ID NO:33-SEQ ID NO:35;
SEQ ID NO:4-SEQ ID NO:29-SEQ ID NO:33-SEQ ID NO:35;
SEQ ID NO:4-SEQ ID NO:30-SEQ ID NO:33-SEQ ID NO:35;
SEQ ID NO:4-SEQ ID NO:31-SEQ ID NO:33-SEQ ID NO:35; or
SEQ ID NO:4-SEQ ID NO:32-SEQ ID NO:33-SEQ ID NO:35.

These embodiments are shown in Table 2.

TABLE 2
(5′ to 3′ order: BoxB-tracrRNA antirepeat-gRNA scaffold-RTTPBS/PBS-RNA
stabilization sequence; wherein the RTT/PBS sequence is optional and may be
substituted with any RTT/PBS specific for the genomic locus of interest)

| | | | | RTT and | |
|---|---|---|---|---|---|
| | | | | PBS: | |
| | | | | Exemplary | |
| | | | | only; | |
| | | | | specific | |
| | | | | for the | |
| | | | | genomic | RNA |
| | | petracrRNA_ | | locus of | stabilization |
| | BoxB | antirepeat | qRNA scaffold | interest | sequence |
| BoxB- | GGAUAGGGCCC | UAGCAAGUU | AAGGCUAGUCCGUUA | UCUGCCAUCA | UUGACGCGGUUCUAU |
| petracrRNA- | UGAAGAAGGGC | UAAAU (SEQ | UCAACUUGAAAAAGU | AAGCGUGCUC | CUAGUUACGCGUUAA |
| GCUA | CCUAUCUCUUC | ID NO: 21) | GGGACCGAGUCGGUC | AGUCUG | ACCAACUAGAAA |
| | (SEQ ID | | C (SEQ ID | (SEQ ID | (SEQ ID NO: 35) |
| | NO: 4) | | NO: 33) | NO: 34) | |
| BoxB- | GGAUAGGGCCC | UAUCAAGUU | AAGGCUAGUCCGUUA | UCUGCCAUCA | UUGACGCGGUUCUAU |
| petracrRNA- | UGAAGAAGGGC | UAAAU (SEQ | UCAACUUGAAAAAGU | AAGCGUGCUC | CUAGUUACGCGUUAA |
| GAUA | CCUAUCUCUUC | ID NO: 22) | GGGACCGAGUCGGUC | AGUCUG | ACCAACUAGAAA |
| | (SEQ ID | | C (SEQ ID | (SEQ ID | (SEQ ID NO: 35) |
| | NO: 4) | | NO: 33) | NO: 34) | |
| BoxB- | GGAUAGGGCCC | UUAUCAAGU | AAGGCUAGUCCGUUA | UCUGCCAUCA | UUGACGCGGUUCUAU |
| petracrRNA- | UGAAGAAGGGC | UUAAAU | UCAACUUGAAAAAGU | AAGCGUGCUC | CUAGUUACGCGUUAA |
| GAUAA | CCUAUCUCUUC | (SEQ ID | GGGACCGAGUCGGUC | AGUCUG | ACCAACUAGAAA |
| | (SEQ ID | NO: 23) | C (SEQ ID | (SEQ ID | (SEQ ID NO: 35) |
| | NO: 4) | | NO: 33) | NO: 34) | |
| BoxB- | GGAUAGGGCCC | UAACAAGUU | AAGGCUAGUCCGUUA | UCUGCCAUCA | UUGACGCGGUUCUAU |
| petracrRNA- | UGAAGAAGGGC | UAAAU (SEQ | UCAACUUGAAAAAGU | AAGCGUGCUC | CUAGUUACGCGUUAA |
| GUUA | CCUAUCUCUUC | ID NO: 24) | GGGACCGAGUCGGUC | AGUCUG | ACCAACUAGAAA |
| | (SEQ ID | | C (SEQ ID | (SEQ ID | (SEQ ID NO: 35) |
| | NO: 4) | | NO: 33) | NO: 34) | |
| BoxB- | GGAUAGGGCCC | GAACAAGUU | AAGGCUAGUCCGUUA | UCUGCCAUCA | UUGACGCGGUUCUAU |
| petracrRNA- | UGAAGAAGGGC | UAAAU (SEQ | UCAACUUGAAAAAGU | AAGCGUGCUC | CUAGUUACGCGUUAA |
| GUUC | CCUAUCUCUUC | ID NO: 25) | GGGACCGAGUCGGUC | AGUCUG | ACCAACUAGAAA |
| | (SEQ ID | | C (SEQ ID | (SEQ ID | (SEQ ID NO: 35) |
| | NO: 4) | | NO: 33) | NO: 34) | |
| BOXB- | GGAUAGGGCCC | GCGCAAGUU | AAGGCUAGUCCGUUA | UCUGCCAUCA | UUGACGCGGUUCUAU |
| petracrRNA- | UGAAGAAGGGC | UAAAU (SEQ | UCAACUUGAAAAAGU | AAGCGUGCUC | CUAGUUACGCGUUAA |
| GCGC | CCUAUCUCUUC | ID NO: 26) | GGGACCGAGUCGGUC | AGUCUG | ACCAACUAGAAA |
| | (SEQ ID | | C (SEQ ID | (SEQ ID | (SEQ ID NO: 35) |
| | NO: 4) | | NO: 33) | NO: 34) | |
| BoxB- | GGAUAGGGCCC | GACGCAAGU | AAGGCUAGUCCGUUA | UCUGCCAUCA | UUGACGCGGUUCUAU |
| petracrRNA- | UGAAGAAGGGC | UUAAAU | UCAACUUGAAAAAGU | AAGCGUGCUC | CUAGUUACGCGUUAA |
| GCGUC | CCUAUCUCUUC | (SEQ ID | GGGACCGAGUCGGUC | AGUCUG | ACCAACUAGAAA |
| | (SEQ ID | NO: 27) | C (SEQ ID | (SEQ ID | (SEQ ID NO: 35) |
| | NO: 4) | | NO: 33) | NO: 34) | |
| BoxB- | GGAUAGGGCCC | GUACGCAAG | AAGGCUAGUCCGUUA | UCUGCCAUCA | UUGACGCGGUUCUAU |
| petracrRNA- | UGAAGAAGGGC | UUUAAAU | UCAACUUGAAAAAGU | AAGCGUGCUC | CUAGUUACGCGUUAA |
| GCGUAC | CCUAUCUCUUC | (SEQ ID | GGGACCGAGUCGGUC | AGUCUG | ACCAACUAGAAA |
| | (SEQ ID | NO: 28) | C (SEQ ID | (SEQ ID | (SEQ ID NO: 35) |
| | NO: 4) | | NO: 33) | NO: 34) | |
| BOXB- | GGAUAGGGCCC | GCUACGCAA | AAGGCUAGUCCGUUA | UCUGCCAUCA | UUGACGCGGUUCUAU |
| petracrRNA- | UGAAGAAGGGC | GUUUAAAU | UCAACUUGAAAAAGU | AAGCGUGCUC | CUAGUUACGCGUUAA |
| GCGUAGC | CCUAUCUCUUC | (SEQ ID | GGGACCGAGUCGGUC | AGUCUG | ACCAACUAGAAA |
| | (SEQ ID | NO:29) | (SEQ ID | (SEQ ID | (SEQ ID NO: 35) |
| | NO: 4) | | NO: 33) | NO: 34) | |
| BoxB- | GGAUAGGGCCC | CGCUACGCA | AAGGCUAGUCCGUUA | UCUGCCAUCA | UUGACGCGGUUCUAU |
| petracrRNA- | UGAAGAAGGGC | AGUUUAAAU | UCAACUUGAAAAAGU | AAGCGUGCUC | CUAGUUACGCGUUAA |
| GCGUAGCG | CCUAUCUCUUC | (SEQ ID | GGGACCGAGUCGGUC | AGUCUG | ACCAACUAGAAA |
| | (SEQ ID | NO: 30) | C (SEQ ID | (SEQ ID | (SEQ ID NO: 35) |
| | NO: 4) | | NO: 33) | NO: 34) | |
| BoxB- | GGAUAGGGCCC | CAGCUACGC | AAGGCUAGUCCGUUA | UCUGCCAUCA | UUGACGCGGUUCUAU |
| petracrRNA- | UGAAGAAGGGC | AAGUUUAAA | UCAACUUGAAAAAGU | AAGCGUGCUC | CUAGUUACGCGUUAA |
| GCGU | CCUAUCUCUUC | U (SEQ ID | GGGACCGAGUCGGUC | AGUCUG | ACCAACUAGAAA |
| AGCU | (SEQ ID | NO: 31) | C (SEQ ID | (SEQ ID | (SEQ ID NO: 35) |
| G | NO: 4) | | NO: 33) | NO: 34 | |
| BoxB- | GGAUAGGGCCC | CAGCUGACG | AAGGCUAGUCCGUUA | UCUGCCAUCA | UUGACGCGGUUCUAU |
| petracrRNA- | UGAAGAAGGGC | CAAGUUUAA | UCAACUUGAAAAAGU | AAGCGUGCUC | CUAGUUACGCGUUAA |
| GCGU | CCUAUCUCUUC | AU (SEQ ID | GGGACCGAGUCGGUC | AGUCUG | ACCAACUAGAAA |
| CAGC | (SEQ ID | NO: 32) | C (SEQ ID | (SEQ ID | (SEQ ID NO: 35) |
| UG | NO: 4) | | NO: 33) | NO: 34) | |

In another embodiment, the petracrRNA component comprises or consists of a nucleic acid sequence of SEQ ID NO:37.

(SEQ ID NO: 37)
GGAUAGGGCCCUGAAGAAGGGCCCUAUCUCUUCGCGCAAGUUUAAAUAA
GGCUAGUCCGUUAUCAACUUGAAAAAGUGGGACCGAGUCGGUCCUCUGC
CAUCAAAGCGUGCUCAGUCUGUUGACGCGGUUCUAUCUAGUUACGCGUU
AAACCAACUAGAAA

In another aspect the disclosure provides nucleic acids encoding the crRNA and/or petracrRNA of any embodiment or combination of embodiments of the disclosure. The nucleic acid sequence may comprise DNA, which may include chemically or biochemically modified, non-natural, or derivatized nucleotide bases. Such nucleic acid sequences may comprise additional sequences useful for promoting expression of the crRNA and/or petracrRNA, including but not limited to polyA sequences, modified Kozak sequences, etc. It will be apparent to those of skill in the art, based on the teachings herein, what nucleic acid sequences will encode the crRNAs and/or petracrRNAs of the disclosure.

Exemplary sequences that encode a crRNA, petracrRNA, or components thereof, and constructs encoding the first and second proteins are provided as shown in Tables 3-4. In some embodiments, constructs encoding the first and second proteins may further comprise a sequence encoding a nuclear localization sequence (NLS).

TABLE 3

| SEQ ID | | SEQ ID | |
|---|---|---|---|
| NO: | RNA Sequence | NO: | DNA sequence |
| 1 | NRNDSASSANCASSSNNYN | | |
| 2 | UUCUCCACAUGAGGAUCACCCAUGUGG | 48 | TTCTCCACATGAGGATCACCCATGTGG |
| 3 | GGGCCCUGAAGAAGGGCCC | 49 | GGGCCCTGAAGAAGGGCCC |
| 4 | GGAUAGGGCCCUGAAGAAGGGCCCUAUCUCUUC | 50 | GGATAGGGCCCTGAAGAAGGGCCCTATCTCTTC |
| 5 | GGAGCAGACGAUAUGGCGUCGCUCC | | GGAGCAGACGATATGGCGTCGCTCC |
| 6 | CCAGAUCUGAGCCUGGGAGCUCUCUGG | 52 | CCAGATCTGAGCCTGGGAGCTCTCTGG |
| 7 | GUUUUAGAGCUA | 53 | GTTTTAGAGCTA |
| 8 | GUUUAAGAGCUA | 54 | GTTTAAGAGCTA |
| 9 | GUUUAAGAGAUA | 55 | GTTTAAGAGATA |
| 10 | GUUUAAGAGAUAA | 56 | GTTTAAGAGATAA |
| 11 | GUUUAAGAGUUA | 57 | GTTTAAGAGTTA |
| 12 | GUUUAAGAGUUC | 58 | GTTTAAGAGTTC |
| 13 | GUUUAAGAGCGC | 59 | GTTTAAGAGCGC |
| 14 | GUUUAAGAGCGUC | 60 | GTTTAAGAGCGTC |
| 15 | GUUUAAGAGCGUAC | 61 | GTTTAAGAGCGTAC |
| 16 | GUUUAAGAGCGUAGC | 62 | GTTTAAGAGCGTAGC |
| 17 | GUUUAAGAGCGUAGCG | 63 | GTTTAAGAGCGTAGCG |
| 18 | GUUUAAGAGCGUAGCUG | 64 | GTTTAAGAGCGTAGCTG |
| 19 | GUUUAAGAGCGUCAGCUG | 65 | GTTTAAGAGCGTCAGCTG |
| 20 | GCGCAAGUUUAAAUAAGGCUAGUCCGUUAUCAACUU | 66 | GCGCAAGTTTAAATAAGGCTAGTCCGTTATCAAC |
| | GAAAAAGUGGGACCGAGUCGGUCC | | TTGAAAAAGTGGGACCGAGTCGGTCC |
| 21 | UAGCAAGUUUAAAU | 67 | TAGCAAGTTTAAAT |
| 22 | UAUCAAGUUUAAAU | 68 | TATCAAGTTTAAAT |
| 23 | UUAUCAAGUUUAAAU | 69 | TTATCAAGTTTAAAT |
| 24 | UAACAAGUUUAAAU | 70 | TAACAAGTTTAAAT |
| 25 | GAACAAGUUUAAAU | 71 | GAACAAGTTTAAAT |
| 26 | GOGCAAGUUUAAAU | 72 | GCGCAAGTTTAAAT |
| 27 | GACGCAAGUUUAAAU | 73 | GACGCAAGTTTAAAT |
| 28 | GUACGCAAGUUUAAAU | 74 | GTACGCAAGTTTAAAT |
| 29 | GCUACGCAAGUUUAAAU | 75 | GCTACGCAAGTTTAAAT |
| 30 | CGCUACGCAAGUUUAAAU | 76 | CGCTACGCAAGTTTAAAT |
| 31 | CAGCUACGCAAGUUUAAAU | 77 | CAGCTACGCAAGTTTAAAT |
| 32 | CAGCUGACGCAAGUUUAAAU | 78 | CAGCTGACGCAAGTTTAAAT |
| 33 | AAGGCUAGUCCGUUAUCAACUUGAAAAAGUGGGACC | 79 | AAGGCTAGTCCGTTATCAACTTGAAAAAGTGGGA |
| | GAGUCGGUCC | | CCGAGTCGGTCC |
| 34 | UCUGCCAUCAAAGCGUGCUCAGUCUG | 80 | TCTGCCATCAAAGCGTGCTCAGTCTG |
| 35 | UUGACGCGGUUCUAUCUAGUUACGCGUUAAACCA | 81 | TTGACGCGGTTCTATCTAGTTACGCGTTAAACCA |
| | ACUAGAAA | | ACTAGAAA |
| 36 | GGCCCAGACUGAGCACGUGAGUUUAAGAGCGCUU | 82 | GGCCCAGACTGAGCACGTGAGTTTAAGAGCGCTT |
| | CUCCACAUGAGGAUCACCCAUGUGGUUGACGCGG | | CTCCACATGAGGATCACCCATGTGGTTGACGCGG |
| | UUCUAUCUAGUUACGCGUUAAACCAACUAGAAA | | TTCTATCTAGTTACGCGTTAAACCAACTAGAAA |
| 37 | GGAUAGGGCCCUGAAGAAGGGCCCUAUCUCUUCG | 83 | GGATAGGGCCCTGAAGAAGGGCCCTATCTCTTCG |
| | CGCAAGUUUAAAUAAGGCUAGUCCGUUAUCAACU | | CGCAAGTTTAAATAAGGCTAGTCCGTTATCAACT |
| | UGAAAAAGUGGGACCGAGUCGGUCCUCUGCCAUC | | TGAAAAAGTGGGACCGAGTCGGTCCTCTGCCATC |
| | AAAGCGUGCUCAGUCUGUUGACGCGGUUCUAUCU | | AAAGCGTGCTCAGTCTGTTGACGCGGTTCTATCT |
| | AGUUACGCGUUAAACCAACUAGAAA | | AGTTACGCGTTAAACCAACTAGAAA |

TABLE 4

| SEQ ID | | |
|---|---|---|
| NO | Sequence | Name |
| 38 | ATGCGGGACCACATGGTGCTGCACGAGAGCGTGAACGCCGCCGGCATCACCTCT | GFP11-NLS-MCP- |
| | GGCGGCTCAAAAAGAACCGCCGACGGCAGCGAATTCGAGTCACCCAAGAAGAAG | IRES-LambdaN- |
| | AGGAAAGTCGGCGGCCCTGCTGCCAAGAGGGTCAAGTTGGACGGAAGCGGAGCC | GFP1-10 |
| | AGCAATTTCACCCAGTTCGTGCTGGTCGACAACGGCGGTACAGGCGATGTGACC | |
| | GTGGCCCCTAGCAACTTCGCCAACGGCGTGGCCGAGTGGATCAGCAGCAACAGC | |
| | AGAAGCCAGGCCTATAAGGTGACATGCAGCGTGCGGCAGTCTTCTGCCCAGAAG | |
| | CGCAAGTACACCATCAAGGTGGAGGTGCCTAAAGTGGCTACCCAAACAGTGGGA | |
| | GGCGTGGAGCTGCCTGTGGCAGCCTGGCGGAGCTACCTGAACATGGAACTCACC | |
| | ATCCCTATCTTCGCCACGAACAGCGACTGTGAGCTGATCGTGAAAGCCATGCAG | |
| | GGCCTGCTGAAGGACGGCAACCCCATCCCTTCTGCCATCGCCGCTAATAGTGGA | |
| | CTGTACTAATGACCCCCCCCCCCTAACGTTACTGGCCGAAGCCGCTTGGAATAA | |
| | GGCCGGTGTGCGTTTGTCTATATGTTATTTTCCACCATATTGCCGTCTTTTGGC | |
| | AATGTGAGGGCCCGGAAACCTGGCCCTGTCTTCTTGACGAGCATTCCTAGGGGT | |
| | CTTTCCCCTCTCGCCAAAGGAATGCAAGGTCTGTTGAATGTCGTGAAGGAAGCA | |
| | GTTCCTCTGGAAGCTTCTTGAAGACAAACAACGTCTGTAGCGACCCTTTGCAGG | |
| | CAGCGGAACCCCCCACCTGGCGACAGGTGCCTCTGCGGCCAAAAGCCACGTGTA | |
| | TAAGATACACCTGCAAAGGCGGCACAACCCCAGTGCCACGTTGTGAGTTGGATA | |
| | GTTGTGGAAAGAGTCAAATGGCTCTCCTCAAGCGTATTCAACAAGGGGCTGAAG | |
| | GATGCCCAGAAGGTACCCCATTGTATGGGATCTGATCTGGGGCCTCGGTGCACA | |
| | TGCTTTACATGTGTTTAGTCGAGGTTAAAAAACGTCTAGGCCCCCCGAACCACG | |
| | GGGACGTGGTTTTCCTTTGAAAAACACGATGATAATATGGCCCCAAAGCTGGCT | |
| | ACAATGGAGCAGAAGCTGATCAGCGAGGAAGATCTGAAGCGCCCCGCCGCTACA | |
| | AAGAAAGCCGGCCAGGCCAAGAAGAAGAAAGGCGGTTCCGCCAGCGGCGGCAGC | |
| | ATGGACGCCCAAACCAGGAGACGGGAACGGAGAGCCGAGAAGCAGGCTCAGTGG | |
| | AAGGCCGCTAATTCTGGAGGATCTAGCGGAGGATCCAAACGGACAGCCGACGGA | |
| | AGCGAGTTCGAGTCACCAAAGAAGAAGCGGAAAGTCTCTGGCGGTTCAAGCGGC | |
| | GGCTCTGAGTTCATGAGCAAGGGCGAGGAACTGTTCACCGGAGTGGTGCCAATC | |
| | CTGGTGGAACTGGACGGCGACGTGAACGGCCACAAGTTCAGCGTCAGAGGCGAA | |
| | GGAGAGGGCGACGCCACAATCGGCAAGCTGACCCTGAAGTTTATCTGCACCACC | |
| | GGCAAGCTCCCCGTGCCCTGGCCCACCCTGGTGACAACCCTGACATACGGCGTT | |
| | CAATGTTTTAGCAGATACCCCGATCACATGAAAAGGCACGACTTCTTCAAGTCC | |
| | GCCATGCCTGAGGGCTACGTGCAGGAGCGGACCATCAGCTTTAAGGACGACGGC | |
| | AAATACAAGACAAGAGCCGTGGTCAAGTTCGAGGGCGACACCCTGGTTAATAGA | |
| | ATCGAGCTGAAGGGCACTGATTTCAAGGAGGACGGCAACATCCTGGGCCACAAG | |
| | CTGGAATACAACTTCAACAGCCACAACGTGTACATCACAGCTGACAAGCAGAAG | |
| | AACGGCATCAAAGCCAATTTCACCGTGCGGCACAACGTGGAAGATGGCAGCGTG | |
| | CAGCTGGCCGATCATTATCAGCAGAACACCCCTATTGGCGATGGACCTGTGCTG | |
| | CTGCCTGACAACCACTACCTGTCCACCCAAACCGTGCTGAGCAAGGACCCCAAC | |
| | GAGAAGGGAACA | |
| 39 | ATGAAACGGACAGCCGACGGAAGCGAGTTCGAGTCACCAAAGAAGAAGCGGAAA | Prime Editor |
| | GTCGACAAGAAGTACAGCATCGGCCTGGACATCGGCACCAACTCTGTGGGCTGG | (PEmax-P2A- |
| | GCCGTGATCACCGACGAGTACAAGGTGCCCAGCAAGAAATTCAAGGTGCTGGGC | hMLH1dn) |
| | AACACCGACCGGCACAGCATCAAGAAGAACCTGATCGGAGCCCTGCTGTTCGAC | |
| | AGCGGCGAAACAGCCGAGGCCACCCGGCTGAAGAGAACCGCCAGAAGAAGATAC | |
| | ACCAGACGGAAGAACCGGATCTGCTATCTGCAAGAGATCTTCAGCAACGAGATG | |
| | GCCAAGGTGGACGACAGCTTCTTCCACAGACTGGAAGAGTCCTTCCTGGTGGAA | |
| | GAGGATAAGAAGCACGAGCGGCACCCCATCTTCGGCAACATCGTGGACGAGGTG | |
| | GCCTACCACGAGAAGTACCCCACCATCTACCACCTGAGAAAGAAACTGGTGGAC | |
| | AGCACCGACAAGGCCGACCTGCGGCTGATCTATCTGGCCCTGGCCCACATGATC | |
| | AAGTTCCGGGGCCACTTCCTGATCGAGGGCGACCTGAACCCCGACAACAGCGAC | |
| | GTGGACAAGCTGTTCATCCAGCTGGTGCAGACCTACAACCAGCTGTTCGAGGAA | |
| | AACCCCATCAACGCCAGCGGCGTGGACGCCAAGGCCATCCTGTCTGCCAGACTG | |
| | AGCAAGAGCAGAAAGCTGGAAAATCTGATCGCCCAGCTGCCCGGCGAGAAGAAG | |
| | AATGGCCTGTTCGGAAACCTGATTGCCCTGAGCCTGGGCCTGACCCCCAACTTC | |
| | AAGAGCAACTTCGACCTGGCCGAGGATGCCAAACTGCAGCTGAGCAAGGACACC | |
| | TACGACGACGACCTGGACAACCTGCTGGCCCAGATCGGCGACCAGTACGCCGAC | |
| | CTGTTTCTGGCCGCCAAGAACCTGTCCGACGCCATCCTGCTGAGCGACATCCTG | |
| | AGAGTGAACACCGAGATCACCAAGGCCCCCCTGAGCGCCTCTATGATCAAGAGA | |
| | TACGACGAGCACCACCAGGACCTGACCCTGCTGAAAGCTCTCGTGCGGCAGCAG | |
| | CTGCCTGAGAAGTACAAAGAGATTTTCTTCGACCAGAGCAAGAACGGCTACGCC | |
| | GGCTACATTGACGGCGGAGCCAGCCAGGAAGAGTTCTACAAGTTCATCAAGCCC | |
| | ATCCTGGAAAAGATGGACGGCACCGAGGAACTGCTCGTGAAGCTGAAGAGAGAG | |
| | GACCTGCTGCGGAAGCAGCGGACCTTCGACAACGGCAGCATCCCCCACCAGATC | |
| | CACCTGGGAGAGCTGCACGCCATTCTGCGGCGGCAGGAAGATTTTTACCCATTC | |
| | CTGAAGGACAACCGGGAAAAGATCGAGAAGATCCTGACCTTCCGCATCCCCTAC | |
| | TACGTGGGCCCTCTGGCCAGGGGAAACAGCAGATTCGCCTGGATGACCAGAAAG | |
| | AGCGAGGAAACCATCACCCCCTGGAACTTCGAGGAAGTGGTGGACAAGGGCGCT | |
| | TCCGCCCAGAGCTTCATCGAGCGGATGACCAACTTCGATAAGAACCTGCCCAAC | |
| | GAGAAGGTGCTGCCCAAGCACAGCCTGCTGTACGAGTACTTCACCGTGTATAAC | |
| | GAGCTGACCAAAGTGAAATACGTGACCGAGGGAATGAGAAAGCCCGCCTTCCTG | |
| | AGCGGCGAGCAGAAAAAGGCCATCGTGGACCTGCTGTTCAAGACCAACCGGAAA | |
| | GTGACCGTGAAGCAGCTGAAAGAGGACTACTTCAAGAAAATCGAGTGCTTCGAC | |
| | TCCGTGGAAATCTCCGGCGTGGAAGATCGGTTCAACGCCTCCCTGGGCACATAC | |
| | CACGATCTGCTGAAAATTATCAAGGACAAGGACTTCCTGGACAATGAGGAAAAC | |
| | GAGGACATTCTGGAAGATATCGTGCTGACCCTGACACTGTTTGAGGACAGAGAG | |
| | ATGATCGAGGAACGGCTGAAAACCTATGCCCACCTGTTCGACGACAAAGTGATG | |
| | AAGCAGCTGAAGCGGCGGAGATACACCGGCTGGGGCAGGCTGAGCCGGAAGCTG | |
| | ATCAACGGCATCCGGGACAAGCAGTCCGGCAAGACAATCCTGGATTTCCTGAAG | |
| | TCCGACGGCTTCGCCAACAGAAACTTCATGCAGCTGATCCACGACGACAGCCTG | |
| | ACCTTTAAAGAGGACATCCAGAAAGCCCAGGTGTCCGGCCAGGGCGATAGCCTG | |
| | CACGAGCACATTGCCAATCTGGCCGGCAGCCCCGCCATTAAGAAGGGCATCCTG | |
| | CAGACAGTGAAGGTGGTGGACGAGCTCGTGAAAGTGATGGGCCGGCACAAGCCC | |
| | GAGAACATCGTGATCGAAATGGCCAGAGAGAACCAGACCACCCAGAAGGGACAG | |
| | AAGAACAGCCGCGAGAGAATGAAGCGGATCGAAGAGGGCATCAAAGAGCTGGGC | |
| | AGCCAGATCCTGAAAGAACACCCCGTGGAAAACACCCAGCTGCAGAACGAGAAG | |
| | CTGTACCTGTACTACCTGCAGAATGGGGGGGATATGTACGTGGACCAGGAACTG | |
| | GACATCAACCGGCTGTCCGACTACGATGTGGACGCTATCGTGCCTCAGAGCTTT | |
| | CTGAAGGACGACTCCATCGACAACAAGGTGCTGACCAGAAGCGACAAGAACCGG | |
| | GGCAAGAGCGACAACGTGCCCTCCGAAGAGGTCGTGAAGAAGATGAAGAACTAC | |
| | TGGCGGCAGCTGCTGAACGCCAAGCTGATTACCCAGAGAAAGTTCGACAATCTG | |
| | ACCAAGGCCGAGAGAGGCGGCCTGAGCGAACTGGATAAGGCCGGCTTCATCAAG | |
| | AGACAGCTGGTGGAAACCCGGCAGATCACAAAGCACGTGGCACAGATCCTGGAC | |
| | TCCCGGATGAACACTAAGTACGACGAGAATGACAAGCTGATCCGGGAAGTGAAA | |
| | GTGATCACCCTGAAGTCCAAGCTGGTGTCCGATTTCCGGAAGGATTTCCAGTTT | |
| | TACAAAGTGCGCGAGATCAACAACTACCACCACGCCCACGACGCCTACCTGAAC | |
| | GCCGTCGTGGGAACCGCCCTGATCAAAAAGTACCCTAAGCTGGAAAGCGAGTTC | |
| | GTGTACGGCGACTACAAGGTGTACGACGTGCGGAAGATGATCGCCAAGAGCGAG | |
| | CAGGAAATCGGCAAGGCTACCGCCAAGTACTTCTTCTACAGCAACATCATGAAC | |
| | TTTTTCAAGACCGAGATTACCCTGGCCAACGGCGAGATCCGGAAGCGGCCTCTG | |
| | ATCGAGACAAACGGCGAAACCGGGGAGATCGTGTGGGATAAGGGCCGGGATTTT | |
| | GCCACCGTGCGGAAAGTGCTGAGCATGCCCCAAGTGAATATCGTGAAAAAGACC | |
| | GAGGTGCAGACAGGCGGCTTCAGCAAAGAGTCTATCCTGCCCAAGAGGAACAGC | |
| | GATAAGCTGATCGCCAGAAAGAAGGACTGGGACCCTAAGAAGTACGGCGGCTTC | |
| | GACAGCCCCACCGTGGCCTATTCTGTGCTGGTGGTGGCCAAAGTGGAAAAGGGC | |
| | AAGTCCAAGAAACTGAAGAGTGTGAAAGAGCTGCTGGGGATCACCATCATGGAA | |
| | AGAAGCAGCTTCGAGAAGAATCCCATCGACTTTCTGGAAGCCAAGGGCTACAAA | |
| | GAAGTGAAAAAGGACCTGATCATCAAGCTGCCTAAGTACTCCCTGTTCGAGCTG | |
| | GAAAACGGCCGGAAGAGAATGCTGGCCTCTGCCGGCGAACTGCAGAAGGGAAAC | |
| | GAACTGGCCCTGCCCTCCAAATATGTGAACTTCCTGTACCTGGCCAGCCACTAT | |
| | GAGAAGCTGAAGGGCTCCCCCGAGGATAATGAGCAGAAACAGCTGTTTGTGGAA | |
| | CAGCACAAGCACTACCTGGACGAGATCATCGAGCAGATCAGCGAGTTCTCCAAG | |
| | AGAGTGATCCTGGCCGACGCTAATCTGGACAAAGTGCTGTCCGCCTACAACAAG | |
| | CACCGGGATAAGCCCATCAGAGAGCAGGCCGAGAATATCATCCACCTGTTTACC | |
| | CTGACCAATCTGGGAGCCCCTGCCGCCTTCAAGTACTTTGACACCACCATCGAC | |
| | CGGAAGAGGTACACCAGCACCAAAGAGGTGCTGGACGCCACCCTGATCCACCAG | |
| | AGCATCACCGGCCTGTACGAGACACGGATCGACCTGTCTCAGCTGGGAGGTGAC | |
| | TCCGGCGGAAGCTCTGGTGGCAGCAAGCGGACCGCCGACGGCTCTGAATTCGAG | |
| | AGCCCTAAGAAGAAAAGAAAGGTGAGCGGAGGCTCTAGCGGCGGAAGCACCCTG | |
| | AACATTGAAGACGAGTATAGACTGCATGAAACAAGCAAGGAACCCGACGTGTCC | |
| | CTGGGCTCCACCTGGCTGTCCGACTTTCCCCAGGCCTGGGCCGAGACAGGAGGA | |
| | ATGGGCCTGGCCGTGCGGCAGGCACCCCTGATCATCCCTCTGAAGGCCACCTCT | |
| | ACACCCGTGAGCATCAAGCAGTACCCTATGTCTCAGGAGGCCAGACTGGGCATC | |
| | AAGCCTCACATCCAGAGGCTGCTGGACCAGGGCATCCTGGTGCCATGCCAGAGC | |
| | CCCTGGAACACACCACTGCTGCCCGTGAAGAAGCCAGGCACCAATGACTATAGA | |
| | CCCGTGCAGGATCTGAGAGAGGTGAACAAGAGGGTGGAGGATATCCACCCCACC | |
| | GTGCCCAACCCTTACAATCTGCTGTCCGGCCTGCCCCCTTCTCACCAGTGGTAT | |
| | ACAGTGCTGGACCTGAAGGATGCCTTCTTTTGTCTGAGACTGCACCCTACCAGC | |
| | CAGCCACTGTTCGCCTTTGAGTGGAGGGACCCTGAGATGGGCATCTCTGGCCAG | |
| | CTGACCTGGACACGCCTGCCTCAGGGCTTCAAGAATAGCCCAACACTGTTTAAC | |
| | GAGGCCCTGCACCGCGACCTGGCAGATTTCCGGATCCAGCACCCAGATCTGATC | |
| | CTGCTGCAGTACGTGGACGATCTGCTGCTGGCCGCCACCAGCGAGCTGGATTGC | |
| | CAGCAGGGAACACGCGCCCTGCTGCAGACCCTGGGAAACCTGGGATATAGGGCA | |
| | TCCGCCAAGAAGGCCCAGATCTGTCAGAAGCAGGTGAAGTACCTGGGCTATCTG | |
| | CTGAAGGAGGGCCAGAGATGGCTGACAGAGGCCAGGAAGGAGACAGTGATGGGC | |
| | CAGCCAACACCCAAGACCCCAAGACAGCTGAGGGAGTTCCTGGGCAAAGCAGGA | |
| | TTTTGCAGGCTGTTCATCCCAGGATTCGCAGAGATGGCAGCACCTCTGTACCCA | |
| | CTGACCAAGCCGGGCACCCTGTTTAATTGGGGCCCTGACCAGCAGAAGGCCTAT | |
| | CAGGAGATCAAGCAGGCCCTGCTGACAGCACCAGCCCTGGGCCTGCCAGACCTG | |
| | ACCAAGCCTTTCGAGCTGTTTGTGGATGAGAAGCAGGGCTACGCCAAGGGCGTG | |
| | CTGACCCAGAAGCTGGGACCATGGAGACGGCCCGTGGCCTATCTGTCCAAGAAG | |
| | CTGGACCCAGTGGCAGCAGGATGGCCACCATGCCTGAGGATGGTGGCAGCAATC | |
| | GCCGTGCTGACAAAGGATGCCGGCAAGCTGACCATGGGACAGCCACTGGTCATC | |
| | CTGGCACCACACGCAGTGGAGGCCCTGGTGAAGCAGCCTCCAGATCGCTGGCTG | |
| | TCTAACGCCCGGATGACACACTACCAGGCCCTGCTGCTGGACACCGATCGCGTG | |
| | CAGTTTGGCCCTGTGGTGGCCCTGAATCCAGCCACCCTGCTGCCTCTGCCAGAG | |
| | GAGGGCCTGCAGCACAACTGTCTGGACATCCTGGCAGAGGCACACGGAACAAGG | |
| | CCAGACCTGACCGATCAGCCCCTGCCTGACGCCGATCACACATGGTATACCGAT | |
| | GGAAGCTCCCTGCTGCAGGAGGGCCAGAGGAAGGCAGGAGCAGCAGTGACCACA | |
| | GAGACAGAAGTGATCTGGGCCAAGGCCCTGCCAGCAGGCACATCCGCCCAGCGG | |
| | GCCGAGCTGATCGCCCTGACCCAGGCCCTGAAGATGGCCGAGGGCAAGAAGCTG | |
| | AACGTGTACACAGACTCCAGATATGCCTTCGCCACCGCACACATCCACGGAGAG | |
| | ATCTACAGGCGCCGGGGCTGGCTGACCTCTGAGGGCAAGGAGATCAAGAACAAG | |
| | GATGAGATCCTGGCCCTGCTGAAGGCCCTGTTTCTGCCCAAGCGGCTGAGCATC | |
| | ATCCACTGTCCTGGACACCAGAAGGGACACTCCGCCGAGGCAAGGGGCAATCGG | |
| | ATGGCCGACCAGGCCGCCAGAAAGGCTGCTATTACTGAAACTCCCGACACTTCC | |
| | ACTCTGCTGATTGAAAACTCCTCCCCTTCTGGCGGCTCAAAAAGAACCGCCGAC | |
| | GGCAGCGAATTCGAGTCTCCCAAGAAGAAGAGGAAAGTCGGCTCTGGCCCTGCC | |
| | GCTAAGAGAGTGAAGCTGGACGGAAGCGGAGCTACTAACTTCAGCCTGCTGAAG | |
| | CAGGCTGGAGACGTGGAGGAGAACCCTGGACCTAGCTTCGTTGCTGGAGTCATC | |
| | CGGAGACTGGACGAGACAGTGGTGAACAGAATTGCCGCCGGCGAGGTGATCCAG | |
| | AGACCTGCCAATGCAATAAAGGAGATGATCGAGAACTGTCTGGACGCCAAGTCC | |
| | ACAAGCATTCAGGTGATCGTGAAGGAGGGCGGACTGAAGCTGATCCAGATCCAA | |
| | GACAACGGCACAGGCATCAGAAAGGAAGATCTGGACATCGTGTGTGAACGGTTC | |
| | ACCACATCTAAGCTGCAGTCTTTTGAGGATCTGGCCTCTATCAGTACCTACGGC | |
| | TTCAGAGGCGAGGCCCTGGCCAGCATCAGCCACGTGGCCCATGTGACCATCACC | |
| | ACCAAAACCGCCGACGGCAAATGCGCTTATCGCGCTAGCTACAGCGACGGCAAG | |
| | CTGAAAGCCCCGCCAAAGCCTTGCGCCGGCAACCAGGGTACACAGATAACAGTG | |
| | GAGGATCTGTTCTACAACATCGCCACCCGGAGAAAGGCCCTGAAAAATCCCAGC | |
| | GAGGAGTACGGCAAGATCCTGGAAGTCGTCGGCAGATACTCCGTGCACAACGCC | |
| | GGAATCAGCTTTAGCGTAAAGAAGCAGGGAGAAACCGTGGCCGATGTGCGCACC | |
| | CTGCCAAATGCCAGCACCGTGGATAACATCAGAAGCATTTTCGGAAATGCCGTG | |
| | TCCAGAGAACTGATCGAGATCGGCTGCGAAGATAAGACCCTGGCTTTTAAGATG | |
| | AACGGCTACATCAGCAACGCCAATTACTCTGTGAAGAAGTGCATCTTTCTTCTG | |
| | TTCATCAACCACAGACTGGTGGAAAGCACCAGCCTGCGGAAAGCCATCGAGACA | |
| | GTGTACGCCGCCTACCTGCCTAAGAACACCCACCCCTTCCTGTACCTGAGCCTC | |
| | GAGATCAGCCCTCAGAACGTGGACGTCAATGTGCATCCTACAAAGCACGAGGTG | |
| | CACTTCCTGCACGAGGAAAGCATCCTGGAAAGAGTGCAGCAGCACATTGAGAGC | |
| | AAGCTGCTGGGCTCTAACAGCAGCAGAATGTACTTCACACAGACCCTGCTGCCT | |
| | GGCCTGGCCGGCCCCTCAGGCGAAATGGTTAAGTCCACAACCTCTCTGACCTCA | |
| | TCCAGCACCAGCGGTTCTTCCGATAAGGTGTACGCCCACCAGATGGTGCGGACC | |
| | GACTCTCGGGAGCAGAAGCTGGACGCCTTTCTGCAACCTCTGAGCAAACCTCTG | |
| | AGCTCTCAGCCTCAGGCCATCGTGACCGAGGACAAGACAGATATCTCCTCCGGC | |
| | CGTGCCAGACAGCAGGACGAAGAAATGCTCGAGCTGCCAGCTCCTGCCGAGGTG | |
| | GCCGCCAAGAACCAGAGCCTGGAGGGAGATACCACAAAGGGCACCAGCGAAATG | |
| | AGCGAGAAGCGGGGCCCTACCTCCAGCAACCCCAGAAAACGGCACCGGGAGGAC | |
| | AGCGACGTGGAAATGGTGGAGGACGACAGCAGAAAGGAAATGACAGCCGCTTGT | |
| | ACCCCTAGAAGAAGAATCATCAACCTGACCTCCGTGCTGAGCCTGCAGGAGGAG | |
| | ATCAACGAGCAGGGCCACGAGGTGCTGAGAGAGATGCTGCACAATCACAGCTTC | |
| | GTGGGCTGCGTGAACCCTCAATGGGCCCTGGCTCAGCATCAAACAAAGCTGTAC | |
| | CTGCTGAACACCACCAAGCTGAGCGAAGAGCTGTTCTACCAGATCCTCATCTAC | |
| | GACTTCGCCAACTTCGGCGTGCTACGCCTGAGCGAGCCCGCCCCTCTGTTTGAC | |
| | CTGGCCATGCTGGCTCTGGATAGCCCAGAAAGCGGCTGGACAGAAGAGGACGGA | |
| | CCTAAAGAGGGGCTGGCTGAATACATCGTGGAGTTCCTGAAGAAAAAGGCCGAG | |
| | ATGCTGGCCGACTACTTTTCTCTGGAAATCGACGAGGAAGGCAACCTGATCGGC | |
| | CTGCCTCTGCTGATCGATAACTACGTGCCTCCCCTGGAAGGCCTGCCCATCTTC | |
| | ATCCTGAGACTGGCTACAGAGGTGAACTGGGACGAGGAAAAGGAATGCTTCGAG | |
| | TCTCTGAGCAAGGAGTGCGCCATGTTCTATAGCATCAGAAAACAGTACATCTCT | |
| | GAAGAGAGCACTCTGTCTGGCCAGCAGAGTGAAGTGCCCGGAAGCATCCCCAAC | |
| | AGCTGGAAGTGGACCGTGGAACACATCGTGTACAAGGCCCTGCGGAGCCACATT | |
| | CTCCCTCCTAAGCACTTCACCGAGGACGGCAACATCCTGCAGCTGGCCAACCTG | |
| | CCCGACCTTTATAAGGTTTTCTAA | |
| 40 | ATGGCCCCAAAGCTGGCTACAGGCGGTTCCGCCAGCGGCGGCAGCATGGACGCC | ScFv-LambdaN |
| | CAAACCAGGAGACGGGAACGGAGAGCCGAGAAGCAGGCTCAGTGGAAGGCCGCT | |
| | AATTCTGGAGGATCTAGCGGAGGATCCATGGGCCCTGATATCGTGATGACCCAG | |
| | AGCCCTAGCTCCCTGTCTGCTTCTGTGGGAGATAGAGTGACCATTACATGTAGA | |
| | AGCTCCACCGGCGCCGTGACAACCAGCAACTACGCCTCTTGGGTCCAGGAGAAG | |
| | CCTGGCAAACTGTTTAAGGGCCTGATCGGAGGAACAAACAACAGAGCCCCAGGC | |
| | GTCCCCAGCCGGTTCAGCGGCAGCCTGATCGGCGATAAGGCCACACTGACCATC | |
| | AGCAGCCTGCAGCCTGAGGACTTCGCCACCTACTTCTGCGCCCTGTGGTACAGC | |
| | AATCACTGGGTGTTCGGCCAGGGCACCAAGGTGGAACTGAAGCGGGGTGGAGGT | |
| | GGTTCTGGCGGTGGGGGGTCAGGAGGCGGAGGATCTAGTGGTGGGGGATCCGAA | |
| | GTAAAGTTGTTGGAGTCCGGTGGTGGCCTGGTGCAGCCCGGCGGCAGCCTGAAA | |
| | CTGAGCTGCGCTGTGTCTGGATTTTCTCTGACAGATTACGGCGTGAACTGGGTT | |
| | AGGCAGGCCCCTGGAAGAGGCCTGGAATGGATCGGCGTTATCTGGGGCGACGGC | |
| | ATCACCGACTACAACAGCGCCCTGAAAGACAGATTCATCATCAGCAAGGACAAT | |
| | GGCAAGAACACCGTGTACCTGCAAATGAGCAAGGTGCGGAGCGACGACACCGCC | |
| | CTGTACTACTGCGTGACAGGACTGTTCGACTATTGGGGACAGGGCACCCTCGTG | |
| | ACCGTGTCCAGCTAA | |
| 41 | ATGGGAGGAGAAGAACTTTTGAGCAAGAATTATCATCTTGAGAACGAAGTGGCT | GCN4-MCP |
| | CGTCTTAAGAAATCAGGAAGCGGAGCCAGCAATTTCACCCAGTTCGTGCTGGTC | |
| | GACAACGGCGGTACAGGCGATGTGACCGTGGCCCCTAGCAACTTCGCCAACGGC | |
| | GTGGCCGAGTGGATCAGCAGCAACAGCAGAAGCCAGGCCTATAAGGTGACATGC | |
| | AGCGTGCGGCAGTCTTCTGCCCAGAAGCGCAAGTACACCATCAAGGTGGAGGTG | |
| | CCTAAAGTGGCTACCCAAACAGTGGGAGGCGTGGAGCTGCCTGTGGCAGCCTGG | |
| | CGGAGCTACCTGAACATGGAACTCACCATCCCTATCTTCGCCACGAACAGCGAC | |
| | TGTGAGCTGATCGTGAAAGCCATGCAGGGCCTGCTGAAGGACGGCAACCCCATC | |
| | CCTTCTGCCATCGCCGCTAATAGTGGACTGTACTAA | |
| 42 | ATGGCCCCAAAGCTGGCTACAGGCGGTTCCGCCAGCGGCGGCAGCATGGACGCC | LambdaN-NbALFA |
| | CAAACCAGGAGACGGGAACGGAGAGCCGAGAAGCAGGCTCAGTGGAAGGCCGCT | |
| | AATTCTGGAGGATCTAGCGGAGGATCCTCTGGCGAAGTGCAGCTGCAGGAGAGC | |
| | GGCGGAGGCCTGGTGCAACCTGGAGGCAGCCTGAGACTGAGCTGCACCGCCAGC | |
| | GGCGTGACCATCTCTGCTCTGAACGCCATGGCCATGGGCTGGTATAGACAGGCC | |
| | CCAGGCGAGCGGCGGGTGATGGTCGCCGCTGTGTCCGAGCGCGGAAATGCCATG | |
| | TACCGGGAAAGCGTGCAGGGCAGATTCACCGTTACAAGAGATTTTACAAACAAG | |
| | ATGGTGTCTCTCCAGATGGACAACCTGAAGCCCGAGGACACCGCCGTGTACTAC | |
| | TGTCACGTGCTGGAAGATAGAGTGGACAGCTTCCACGACTACTGGGGCCAGGGC | |
| | ACCCAGGTGACAGTGTCCAGCGGCGCCCCTGGCTTCAGCAGCATCAGCGCCTAA | |
| 43 | ATGGGAGGACCCACCAGACTGGAAGAGGAACTGAGACGGAGACTGACCGAGCCT | ALFA-MCP |
| | GGCTCTGGCGGCTCAGGAAGCGGAGCCAGCAATTTCACCCAGTTCGTGCTGGTC | |
| | GACAACGGCGGTACAGGCGATGTGACCGTGGCCCCTAGCAACTTCGCCAACGGC | |
| | GTGGCCGAGTGGATCAGCAGCAACAGCAGAAGCCAGGCCTATAAGGTGACATGC | |
| | AGCGTGCGGCAGTCTTCTGCCCAGAAGCGCAAGTACACCATCAAGGTGGAGGTG | |
| | CCTAAAGTGGCTACCCAAACAGTGGGAGGCGTGGAGCTGCCTGTGGCAGCCTGG | |
| | CGGAGCTACCTGAACATGGAACTCACCATCCCTATCTTCGCCACGAACAGCGAC | |
| | TGTGAGCTGATCGTGAAAGCCATGCAGGGCCTGCTGAAGGACGGCAACCCCATC | |
| | CCTTCTGCCATCGCCGCTAATAGTGGACTGTACTAA | |
| 44 | ATGGAGCAGAAGCTGATCAGCGAGGAAGATCTGAAGCGCCCCGCCGCTACAAAG | LambdaN-FRB |
| | AAAGCCGGCCAGGCCAAGAAGAAGAAAGGCGGTTCCGCCAGCGGCGGCAGCATG | |
| | GACGCCCAAACCAGGAGACGGGAACGGAGAGCCGAGAAGCAGGCTCAGTGGAAG | |
| | GCCGCTAATTCTGGAGGATCTAGCGGAGGATCCAAACGGACAGCCGACGGAAGC | |
| | GAGTTCGAGTCACCAAAGAAGAAGCGGAAAGTCTCTGGCGGTTCAAGCGGCGGC | |
| | TCTATCCTGTGGCATGAGATGTGGCACGAGGGCCTGGAAGAGGCCTCTAGACTG | |
| | TATTTCGGCGAGCGGAACGTCAAGGGAATGTTCGAGGTGCTGGAACCACTGCAC | |
| | GCCATGATGGAAAGAGGCCCTCAGACCCTGAAGGAAACCAGCTTTAACCAGGCC | |
| | TACGGCAGAGATCTGATGGAAGCTCAGGAGTGGTGCAGAAAGTACATGAAAAGC | |
| | GGCAACGTGAAGGACCTGACACAGGCCTGGGACCTCTACTACCACGTGTTCAGA | |
| | AGAATCTCTAAGTAA | |
| 45 | ATGGGCGTCCAGGTGGAAACCATCAGCCCTGGAGATGGCAGAACCTTCCCCAAG | FKBP-MCP |
| | CGGGGCCAGACCTGCGTGGTGCACTACACAGGCATGCTGGAAGATGGAAAGAAA | |
| | TTTGACAGCTCCAGAGATAGAAACAAGCCTTTTAAGTTCATGCTGGGCAAGCAG | |
| | GAGGTGATCAGAGGCTGGGAGGAAGGCGTTGCTCAGATGAGCGTGGGCCAAAGA | |
| | GCCAAGCTGACCATTTCTCCCGACTACGCCTACGGCGCCACAGGCCACCCCGGA | |
| | ATCATCCCACCTCACGCCACCCTGGTGTTCGACGTGGAGCTGCTGAAACTGGAA | |
| | TCTGGCGGCTCAAAAAGAACCGCCGACGGCAGCGAATTCGAGTCACCCAAGAAG | |
| | AAGAGGAAAGTCGGCGGCCCTGCTGCCAAGAGGGTCAAGTTGGACGGAAGCGGA | |
| | GCCAGCAATTTCACCCAGTTCGTGCTGGTCGACAACGGCGGTACAGGCGATGTG | |
| | ACCGTGGCCCCTAGCAACTTCGCCAACGGCGTGGCCGAGTGGATCAGCAGCAAC | |
| | AGCAGAAGCCAGGCCTATAAGGTGACATGCAGCGTGCGGCAGTCTTCTGCCCAG | |
| | AAGCGCAAGTACACCATCAAGGTGGAGGTGCCTAAAGTGGCTACCCAAACAGTG | |
| | GGAGGCGTGGAGCTGCCTGTGGCAGCCTGGCGGAGCTACCTGAACATGGAACTC | |
| | ACCATCCCTATCTTCGCCACGAACAGCGACTGTGAGCTGATCGTGAAAGCCATG | |
| | CAGGGCCTGCTGAAGGACGGCAACCCCATCCCTTCTGCCATCGCCGCTAATAGT | |
| | GGACTGTACTAA | |
| 46 | ATGGCCCCAAAGCTGGCTACAGGCGGTTCCGCCAGCGGCGGCAGCATGGACGCC | LambdaN-ABI |
| | CAAACCAGGAGACGGGAACGGAGAGCCGAGAAGCAGGCTCAGTGGAAGGCCGCT | |
| | AATTCTGGAGGATCTAGCGGAGGATCCACAAGAGTGCCTCTGTACGGCTTCACC | |
| | AGCATCTGCGGACGGCGCCCCGAGATGGAAGCCGCCGTTTCTACCATCCCAAGA | |
| | TTTCTGCAGAGCAGCTCCGGCAGCATGCTGGACGGCAGATTCGACCCCCAGTCC | |
| | GCCGCACATTTCTTCGGCGTGTACGACGGCCACGGCGGCTCTCAGGTGGCTAAT | |
| | TACTGCAGAGAGCGGATGCACCTGGCCCTGGCCGAGGAAATCGCCAAGGAGAAG | |
| | CCCATGCTCTGTGACGGAGATACATGGCTGGAAAAGTGGAAGAAGGCCCTGTTC | |
| | AACAGCTTCCTGAGAGTTGACAGCGAGATCGAGAGCGTGGCCCCTGAAACCGTG | |
| | GGCAGCACCTCCGTGGTGGCTGTGGTCTTTCCCAGCCACATCTTCGTGGCCAAC | |
| | TGCGGCGATTCTCGGGCCGTGCTGTGTAGAGGCAAGACAGCCCTGCCTCTGTCC | |
| | GTGGACCACAAACCTGACCGGGAAGATGAGGCCGCCCGGATCGAGGCCGCTGGT | |
| | GGAAAGGTGATCCAGTGGAACGGCGCCAGGGTGTTCGGCGTGCTGGCCATGAGC | |
| | AGAAGCATCGGCGACAGATATCTGAAACCTAGCATTATCCCTGATCCAGAGGTG | |
| | ACCGCCGTCAAGCGGGTGAAGGAAGATGACTGCCTGATCCTGGCTTCTGATGGC | |
| | GTGTGGGACGTGATGACCGACGAGGAGGCCTGCGAGATGGCCAGAAAGAGAATC | |
| | CTGCTGTGGCACAAGAAAAACGCCGTGGCCGGCGACGCCAGCCTGCTGGCTGAT | |
| | GAGAGAAGAAAGGAAGGCAAAGACCCTGCCGCTATGAGCGCCGCTGAATACCTG | |
| | AGCAAGCTGGCCATCCAAAGAGGATCTAAGGACAACATCAGCGTGGTGGTGGTG | |
| | GACCTGAAGTAA | |
| 47 | ATGGGAGCTCCTACCCAAGACGAGTTCACCCAGCTGAGCCAGAGCATCGCCGAG | PYL-MCP |
| | TTTCACACATACCAGCTGGGCAACGGCAGATGTTCCAGCCTGCTGGCCCAGAGA | |
| | ATCCACGCCCCTCCAGAAACCGTGTGGAGCGTGGTGCGGAGGTTCGACAGACCC | |
| | CAGATCTATAAGCACTTCATCAAGAGCTGCAACGTGTCCGAGGACTTCGAGATG | |
| | AGAGTGGGCTGCACACGGGACGTGAACGTGATCAGCGGCCTGCCTGCCAATACC | |
| | AGCAGAGAGCGGCTGGATCTGCTGGACGATGACCGGGGGGTGACAGGCTTCAGC | |
| | ATCACCGGAGGCGAGCACCGGCTCAGAAACTACAAGTCTGTGACCACCGTGCAT | |
| | AGATTTGAGAAAGAGGAAGAAGAGGAAAGAATCTGGACCGTCGTCCTGGAAAGC | |
| | TACGTGGTTGACGTGCCCGAGGGCAATTCTGAAGAAGATACAAGACTGTTCGCC | |
| | GATACCGTGATCAGACTGAACCTGCAGAAGCTGGCTTCTATTACAGAGGCCATG | |
| | AACGGCTCTGGCGGCTCAGGAAGCGGAGCCAGCAATTTCACCCAGTTCGTGCTG | |
| | GTCGACAACGGCGGTACAGGCGATGTGACCGTGGCCCCTAGCAACTTCGCCAAC | |
| | GGCGTGGCCGAGTGGATCAGCAGCAACAGCAGAAGCCAGGCCTATAAGGTGACA | |
| | TGCAGCGTGCGGCAGTCTTCTGCCCAGAAGCGCAAGTACACCATCAAGGTGGAG | |
| | GTGCCTAAAGTGGCTACCCAAACAGTGGGAGGCGTGGAGCTGCCTGTGGCAGCC | |
| | TGGCGGAGCTACCTGAACATGGAACTCACCATCCCTATCTTCGCCACGAACAGC | |
| | GACTGTGAGCTGATCGTGAAAGCCATGCAGGGCCTGCTGAAGGACGGCAACCCC | |
| | ATCCCTTCTGCCATCGCCGCTAATAGTGGACTGTACTAA | |
| 84 | (GGCCCAGACTGAGCACGTGA)GTTTAAGAGCTATTCTCCACATGAGGATCACC | Full length DNA |
| | CATGTGG[TTGACGCGGTTCTATCTAGTTACGCGTTAAACCAACTAGAAA] | sequence encoding |
| | | crRNA-MS2-GCUA |
| | | where sequence in |
| | | ( ) encodes the |
| | | CRISPR spacer |
| | | sequence and the |
| | | sequence in [ ] |
| | | encodes RNA |
| | | stabilization |
| | | sequence. |
| 85 | (GGCCCAGACTGAGCACGTGA)GTTTAAGAGATATTCTCCACATGAGGATCACC | Full length DNA |
| | CATGTGG[TTGACGCGGTTCTATCTAGTTACGCGTTAAACCAACTAGAAA] | sequence encoding |
| | | crRNA-MS2-GAUA |
| | | where sequence in |
| | | ( ) encodes the |
| | | CRISPR spacer |
| | | sequence and the |
| | | sequence in [ ] |
| | | encodes RNA |
| | | stabilization |
| | | sequence. |
| 86 | (GGCCCAGACTGAGCACGTGA)GTTTAAGAGATAATTCTCCACATGAGGATCAC | Full length DNA |
| | CCATGTGG[TTGACGCGGTTCTATCTAGTTACGCGTTAAACCAACTAGAAA] | sequence encoding |
| | | crRNA-MS2-GAUAA |
| | | where sequence in |
| | | ( ) encodes the |
| | | CRISPR spacer |
| | | sequence and the |
| | | sequence in [ ] |
| | | encodes RNA |
| | | stabilization |
| | | sequence. |
| 87 | (GGCCCAGACTGAGCACGTGA)GTTTAAGAGTTATTCTCCACATGAGGATCACC | Full length DNA |
| | CATGTGG[TTGACGCGGTTCTATCTAGTTACGCGTTAAACCAACTAGAAA] | sequence encoding |
| | | crRNA-MS2-GUUA |
| | | where sequence in |
| | | ( ) encodes the |
| | | CRISPR spacer |
| | | sequence and the |
| | | sequence in [ ] |
| | | encodes RNA |
| | | stabilization |
| | | sequence. |
| 88 | (GGCCCAGACTGAGCACGTGA)GTTTAAGAGTTCTTCTCCACATGAGGATCACC | Full length DNA |
| | CATGTGG[TTGACGCGGTTCTATCTAGTTACGCGTTAAACCAACTAGAAA] | sequence encoding |
| | | crRNA-MS2-GUUC |
| | | where sequence in |
| | | ( ) encodes the |
| | | CRISPR spacer |
| | | sequence and the |
| | | sequence in [ ] |
| | | encodes RNA |
| | | stabilization |
| | | sequence. |
| 89 | (GGCCCAGACTGAGCACGTGA)GTTTAAGAGCGCTTCTCCACATGAGGATCACC | Full length DNA |
| | CATGTGG[TTGACGCGGTTCTATCTAGTTACGCGTTAAACCAACTAGAAA] | sequence encoding |
| | | crRNA-MS2-GCGC |
| | | where sequence in |
| | | ( ) encodes the |
| | | CRISPR spacer |
| | | sequence and the |
| | | sequence in [ ] |
| | | encodes RNA |
| | | stabilization |
| | | sequence. |
| 90 | (GGCCCAGACTGAGCACGTGA)GTTTAAGAGCGTCTTCTCCACATGAGGATCAC | Full length DNA |
| | CCATGTGG[TTGACGCGGTTCTATCTAGTTACGCGTTAAACCAACTAGAAA] | sequence encoding |
| | | crRNA-MS2-GCGUC |
| | | where sequence in |
| | | ( ) encodes the |
| | | CRISPR spacer |
| | | sequence and the |
| | | sequence in [ ] |
| | | encodes RNA. |
| | | stabilization |
| | | sequence. |
| 91 | (GGCCCAGACTGAGCACGTGA)GTTTAAGAGCGTACTTCTCCACATGAGGATCA | Full length DNA |
| | CCCATGTGG[TTGACGCGGTTCTATCTAGTTACGCGTTAAACCAACTAGAAA] | sequence encoding |
| | | crRNA-MS2-GCGUAC |
| | | where sequence in |
| | | ( ) encodes the |
| | | CRISPR spacer |
| | | sequence and the |
| | | sequence in [ ] |
| | | encodes RNA |
| | | stabilization |
| | | sequence. |
| 92 | (GGCCCAGACTGAGCACGTGA)GTTTAAGAGCGTAGCTTCTCCACATGAGGATC | Full length DNA |
| | ACCCATGTGG[TTGACGCGGTTCTATCTAGTTACGCGTTAAACCAACTAGAAA] | sequence encoding |
| | | crRNA-MS2-GCGUAGC |
| | | where sequence in |
| | | ( ) encodes the |
| | | CRISPR spacer |
| | | sequence and the |
| | | sequence in [ ] |
| | | encodes PNA |
| | | stabilization |
| | | sequence. |
| 93 | (GGCCCAGACTGAGCACGTGA)GTTTAAGAGCGTAGCGTTCTCCACATGAGGAT | Full length DNA |
| | CACCCATGTGG[TTGACGCGGTTCTATCTAGTTACGCGTTAAACCAACTAGAAA] | sequence encoding |
| | | GCGUAGCG where |
| | | sequence in ( ) |
| | | encodes the |
| | | CRISPR spacer |
| | | sequence and the |
| | | sequence in [ ] |
| | | encodes RNA |
| | | stabilization |
| | | sequence. |
| 94 | (GGCCCAGACTGAGCACGTGA)GTTTAAGAGCGTAGCTGTTCTCCACATGAGGA | Full length DNA |
| | TCACCCATGTGG[TTGACGCGGTTCTATCTAGTTACGCGTTAAACCAACTAGAA | sequence encoding |
| | A] | crRNA-MS2- |
| | | GCGUAGCUG where |
| | | sequence in ( ) |
| | | encodes the |
| | | CRISPR spacer |
| | | sequence and the |
| | | sequence in [ ] |
| | | encodes RNA |
| | | stabilization |
| | | sequence. |
| 95 | (GGCCCAGACTGAGCACGTGA)GTTTAAGAGCGTCAGCTGTTCTCCACATGAGG | Full length DNA |
| | ATCACCCATGTGG[TTGACGCGGTTCTATCTAGTTACGCGTTAAACCAACTAGA | sequence encoding |
| | AA] | CERNA-MS2- |
| | | GCGUCAGCUG where |
| | | sequence in ( ) |
| | | encodes the |
| | | CRISPR spacer |
| | | sequence and the |
| | | sequence in [ ] |
| | | encodes RNA. |
| | | stabilization |
| | | sequence. |
| 96 | GGATAGGGCCCTGAAGAAGGGCCCTATCTCTTCTAGCAAGTTTAAATAAGGCTA | Full length DNA |
| | GTCCGTTATCAACTTGAAAAAGTGGGACCGAGTCGGTCC(TCTGCCATCAAAGC | sequence encoding |
| | GTGCTCAGTCTG)[TTGACGCGGTTCTATCTAGTTACGCGTTAAACCAACTAGA | BoxB-petracrRNA- |
| | AA] | GCUA where |
| | | sequence in ( ) |
| | | encodes the |
| | | optional PBS + RTS |
| | | and the sequence |
| | | in [ ] encodes RNA |
| | | stabilization |
| | | sequence. |
| 97 | GGATAGGGCCCTGAAGAAGGGCCCTATCTCTTCTATCAAGTTTAAATAAGGCTA | Full length DNA |
| | GTCCGTTATCAACTTGAAAAAGTGGGACCGAGTCGGTCC(TCTGCCATCAAAGC | sequence encoding |
| | GTGCTCAGTCTG)[TTGACGCGGTTCTATCTAGTTACGCGTTAAACCAACTAGA | BoxB-petracrRNA- |
| | AA] | GAUA where |
| | | sequence in ( ) |
| | | encodes the |
| | | optional PBS + RTS |
| | | and the sequence |
| | | in [ ] encodes RNA |
| | | stabilization |
| | | sequence. |
| 98 | GGATAGGGCCCTGAAGAAGGGCCCTATCTCTTCTTATCAAGTTTAAATAAGGCT | Full length DNA |
| | AGTCCGTTATCAACTTGAAAAAGTGGGACCGAGTCGGTCC(TCTGCCATCAAAG | sequence encoding |
| | CGTGCTCAGTCTG)[TTGACGCGGTTCTATCTAGTTACGCGTTAAACCAACTAG | BoxB-petracrRNA- |
| | AAA] | GAUAA where |
| | | sequence in ( ) |
| | | encodes the |
| | | optional PBS + RTS |
| | | and the sequence |
| | | in [ ] encodes RNA |
| | | stabilization |
| | | sequence. |
| 99 | GGATAGGGCCCTGAAGAAGGGCCCTATCTCTTCTAACAAGTTTAAATAAGGCTA | Full length DNA |
| | GTCCGTTATCAACTTGAAAAAGTGGGACCGAGTCGGTCC(TCTGCCATCAAAGC | sequence encoding |
| | GTGCTCAGTCTG)[TTGACGCGGTTCTATCTAGTTACGCGTTAAACCAACTAGA | BoxB-petracrRNA- |
| | AA] | GUUA where |
| | | sequence in ( ) |
| | | encodes the |
| | | optional PBS + RTS |
| | | and the sequence |
| | | in [ ] encodes RNA |
| | | stabilization |
| | | sequence. |
| 100 | GGATAGGGCCCTGAAGAAGGGCCCTATCTCTTCGAACAAGTTTAAATAAGGCTA | Full length DNA |
| | GTCCGTTATCAACTTGAAAAAGTGGGACCGAGTCGGTCC(TCTGCCATCAAAGC | sequence encoding |
| | GTGCTCAGTCTG)[TTGACGCGGTTCTATCTAGTTACGCGTTAAACCAACTAGA | BoxB-petracrRNA- |
| | AA] | GUUC where |
| | | sequence in ( ) |
| | | encodes the |
| | | optional PBS + RTS |
| | | and the sequence |
| | | in [ ] encodes RNA |
| | | stabilization |
| | | sequence. |
| 101 | GGATAGGGCCCTGAAGAAGGGCCCTATCTCTTCGCGCAAGTTTAAATAAGGCTA | Full length DNA |
| | GTCCGTTATCAACTTGAAAAAGTGGGACCGAGTCGGTCC(TCTGCCATCAAAGC | sequence encoding |
| | GTGCTCAGTCTG)[TTGACGCGGTTCTATCTAGTTACGCGTTAAACCAACTAGA | BozB-petracrRNA- |
| | AA] | GCGC where |
| | | sequence in ( ) |
| | | encodes the |
| | | optional PBS + RTS |
| | | and the sequence |
| | | in [ ] encodes RNA |
| | | stabilization |
| | | sequence. |
| 102 | GGATAGGGCCCTGAAGAAGGGCCCTATCTCTTCGACGCAAGTTTAAATAAGGCT | Full length DNA |
| | AGTCCGTTATCAACTTGAAAAAGTGGGACCGAGTCGGTCC(TCTGCCATCAAAG | sequence encoding |
| | CGTGCTCAGTCTG)[TTGACGCGGTTCTATCTAGTTACGCGTTAAACCAACTAG | BoxB-petracrRNA- |
| | AAA] | GCGUC where |
| | | sequence in ( ) |
| | | encodes the |
| | | optional PBS + RTS |
| | | and the sequence |
| | | in [ ] encodes RNA |
| | | stabilization |
| | | sequence. |
| 103 | GGATAGGGCCCTGAAGAAGGGCCCTATCTCTTCGTACGCAAGTTTAAATAAGGC | Full length DNA |
| | TAGTCCGTTATCAACTTGAAAAAGTGGGACCGAGTCGGTCC(TCTGCCATCAAA | sequence encoding |
| | GCGTGCTCAGTCTG)[TTGACGCGGTTCTATCTAGTTACGCGTTAAACCAACTA | BoxB-petracrRNA- |
| | GAAA] | GCGUAC where |
| | | sequence in ( ) |
| | | encodes the |
| | | optional PBS + RTS |
| | | and the sequence |
| | | in [ ] encodes RNA |
| | | stabilization |
| | | sequence. |
| 104 | GGATAGGGCCCTGAAGAAGGGCCCTATCTCTTCGCTACGCAAGTTTAAATAAGG | Full length DNA |
| | CTAGTCCGTTATCAACTTGAAAAAGTGGGACCGAGTCGGTCC(TCTGCCATCAA | sequence encoding |
| | AGCGTGCTCAGTCTG)[TTGACGCGGTTCTATCTAGTTACGCGTTAAACCAACT | BozB-petracrRNA- |
| | AGAAA] | GCGUAGC where |
| | | sequence in ( ) |
| | | encodes the |
| | | optional PBS + RTS |
| | | and the sequence |
| | | in [ ] encodes RNA |
| | | stabilization |
| | | sequence. |
| 105 | GGATAGGGCCCTGAAGAAGGGCCCTATCTCTTCCGCTACGCAAGTTTAAATAAG | Full length DNA |
| | GCTAGTCCGTTATCAACTTGAAAAAGTGGGACCGAGTCGGTCC(TCTGCCATCA | sequence encoding |
| | AAGCGTGCTCAGTCTG)[TTGACGCGGTTCTATCTAGTTACGCGTTAAACCAAC | BoxB-petracrRNA- |
| | TAGAAA] | GCGUAGCG where |
| | | sequence in ( ) |
| | | encodes the |
| | | optional PBS + RTS |
| | | and the sequence |
| | | in [ ] encodes RNA |
| | | stabilization |
| | | sequence. |
| 106 | GGATAGGGCCCTGAAGAAGGGCCCTATCTCTTCCAGCTACGCAAGTTTAAATAA | Full length DNA |
| | GGCTAGTCCGTTATCAACTTGAAAAAGTGGGACCGAGTCGGTCC(TCTGCCATC | sequence encoding |
| | AAAGCGTGCTCAGTCTG)[TTGACGCGGTTCTATCTAGTTACGCGTTAAACCAA | BoxB-petracrRNA- |
| | CTAGAAA] | GCGUAGCUG where |
| | | sequence in ( ) |
| | | encodes the |
| | | optional PBS + RTS |
| | | and the sequence |
| | | in [ ] encodes RNA |
| | | stabilization |
| | | sequence. |
| 107 | GGATAGGGCCCTGAAGAAGGGCCCTATCTCTTCCAGCTGACGCAAGTTTAAATA | Full length DNA |
| | AGGCTAGTCCGTTATCAACTTGAAAAAGTGGGACCGAGTCGGTCC(TCTGCCAT | sequence encoding |
| | CAAAGCGTGCTCAGTCTG)[TTGACGCGGTTCTATCTAGTTACGCGTTAAACCA | BoxB-petracrRNA- |
| | ACTAGAAA] | GCGUCAGCUG where |
| | | sequence in () |
| | | encodes the |
| | | optional PBS + RTS |
| | | and the sequence |
| | | in [ ] encodes RNA |
| | | stabilization |
| | | sequence. |

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the nucleic acids, expression vectors (i.e.: episomal or chromosomally integrated), and/or split pegRNAs disclosed herein, wherein the host cells can be either prokaryotic or eukaryotic. The cells can be transiently or stably engineered to incorporate the nucleic acids or expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In another embodiment, the disclosure provides kits comprising the split-pegRNA of any embodiment herein. In another embodiment, the disclosure provides kits comprising the nucleic acid, expression vector, and/or host cell of any embodiment herein. The kits may be used, for example, in the methods of the disclosure. The kits may contain any other components that are suitable for an intended purpose. In some embodiments, the kits may further comprise one or more of the following:
(a) a first protein as disclosed in any embodiment herein;
(b) a second protein as disclosed in any embodiment herein; and/or
(c) a modified or unmodified CRISPR-Cas protein as disclosed in any embodiment herein.

The kit can also optionally comprise various buffers and reagents to facilitate the reactions described herein. For example, the kit can comprise dNTPs, RNase inhibitors, cofactors, etc. Each of the components of the kits, where applicable, can be provided in liquid form (e.g., a solution) or solid form (e.g., powdered or lyophilized). In some embodiments some of the components may be reconstitute able or processable, for example by the addition of a suitable solvent.

In another aspect, the disclosure provides methods for recording a protein-protein and/or protein-RNA interaction within a cell, comprising expressing in the cell:
(a) the split pegRNA as disclosed in any embodiment herein,
(b) the first and, if necessary, the second protein as disclosed in any embodiment herein, and
(c) the modified or unmodified CRISPR-Cas protein as disclosed in any embodiment herein;
wherein dimerization of the first and second RNA extensions of the split pegRNA via binding to the first and, if necessary, the second protein in the cell induces formation of functioning guide RNA for genome or epigenome editing via the CRISPR-Cas protein, thereby inducing genome or epigenome editing to edit genomic DNA in the cell and recording of the protein-protein and/or protein-RNA interaction into genomic DNA in the cell.

The cell in which embodiments of the present disclosure are expressed can be any cell. In some embodiments, the cell is a prokaryotic cell. In other embodiments, the cell is a eukaryotic cell, such as without limitation an animal or plant cell. In certain embodiments, the cell is a mammalian cell. As used herein, the term “eukaryotic cell” may refer to a cell or a plurality of cells derived from a eukaryotic organism. In some embodiments, the eukaryotic cells can be derived from an animal (e.g., primate, rodent, mouse, rat, rabbit, canine, dog, cow, bovine, sheep, ovine, goat, pig, fowl, poultry, chicken, fish, insect, or arthropod). In other embodiments, the eukaryotic cells can be derived from a rodent (e.g., mouse). In still other embodiments, the eukaryotic cells can be non-human eukaryotic cells. In other embodiments, eukaryotic cells can be primary cells or cell lines that are well known to one of ordinary skill in the art. In still other embodiments, eukaryotic cells can be dividing cells (e.g., stem cells) or partially or terminally differentiated cells. In other embodiments, eukaryotic cells may in certain embodiments be disease cells (e.g., tumor cells).

### EXAMPLES

### Introduction

Here we present a strategy named “Split-pegRNA recorders”, where specific molecular interactions within cells induce precise genome editing that inserts an event-specific barcode sequence into the predetermined genomic locus of DNA Tape. The molecular interaction between two protein or RNA molecules is sensed by two adaptor modules attached to dual-RNA-guide molecules⁴, similar to crRNA and tracrRNA discovered in the CRISPR-Cas9 systems. The dimerization of adaptor modules induces the formation of functioning guide RNA for prime editing, inducing prime editing to record its occurrence onto the stable genomic DNA medium. We demonstrate that genome editing efficiency depends on the strength of dimerization interactions, therefore faithfully measuring the interaction strength between two molecules.

### Results

Testing Strategies to Design Split-pegRNA with Dimerization Domains

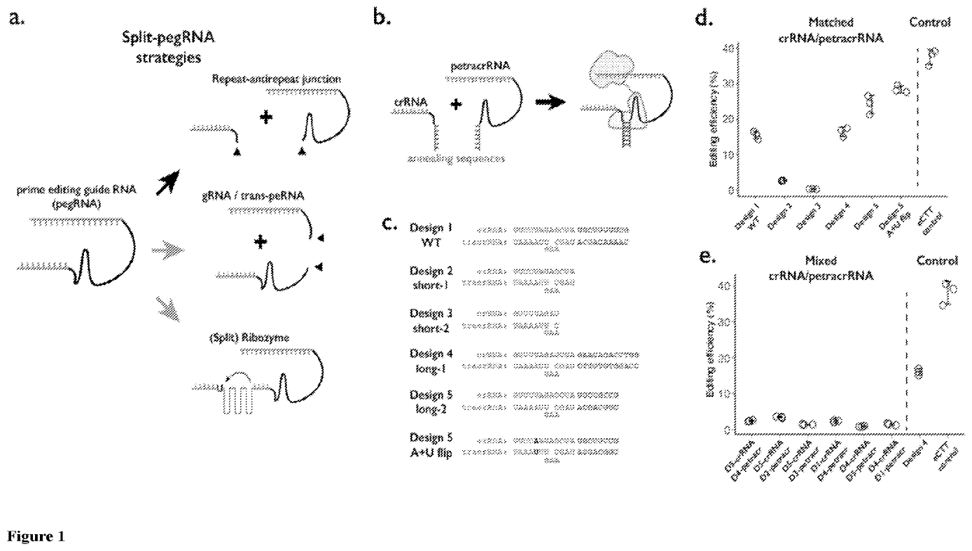

To record the physical interaction between two molecules within the living cell, we designed a precision genome editing system based on prime editing, where we split the prime editing guide RNA (pegRNA) into two complementary parts that are functional only if they form a stable heterodimer. We considered three broad strategies to design two-part pegRNA and engineer the induction of its dimerization (FIG. 1a). First, we generated a dual-RNA-guided system by splitting pegRNA within the sgRNA scaffold, which was originally generated by ligating crRNA and tracrRNA through the GAAA RNA tetraloop⁴. In our case, tracrRNA would be extended with the prime editing-specific reverse-transcription template, referred to as “petracrRNA” (prime editing trans-activating CRISPR RNA). Second, pegRNA can be split into functional sgRNA and reverse-transcription template RNA that may act in trans. Third, we considered inserting a split-ribozyme that splices itself out upon dimerization (e.g., a two-part self-splicing ribozyme from Tetrahymena Thermophilus⁵), irreversibly forming a single pegRNA molecule.

We first tested the prime editing using pegRNAs split at the repeat-antirepeat junction, forming crRNA and petracrRNA molecules. Here, the petracrRNA carries the 3′-end extension necessary for the prime editing (primer binding sites and RT-template sequences for generating the 3′ overhang on the nicked genomic DNA). The upper stem-loop of the repeat-antirepeat region of gRNA is not necessary for Cas9 function and is often omitted within the standard sgRNA constructs. We have replaced it with the two RNA extensions that are reverse-complementary to each other. To inhibit the early degradation of crRNA and petracrRNA, we placed an additional RNA pseudoknot structure at the end of both molecules, a strategy used to form the enhanced pegRNA or “epegRNA” for higher prime editing efficiency⁶. We observed a prime editing efficiency ranging between 15 and 28%, comparable to the editing efficiency achieved by a single epegRNA construct (37%). The editing is specific to the RNA-RNA annealing sequence; constructs lacking or incompatible annealing sequences exhibited markedly lower editing efficiency (0.5-3%) (FIG. 1d,e), potentially making this approach attractive for developing Split-pegRNA recorders.

Next, as an independent approach for developing Split-pegRNA recorders, we tested prime editing using sgRNA and trans-prime-editing RNA, where pegRNA is split at its 3′ extension junction (FIG. 1a, middle). In this strategy, dimerization of two RNA molecules is driven by the annealing reverse-complementary of RNA sequences. We tested a handful of dimerization sequences that range its melting temperature from 45° C. to 75° C., but observed a low editing efficiency (<2%) in general. The inefficiency in editing is less likely due to the inserted dimerization domain because a single pegRNA with additional RNA stem-loop structure at the PE-junction exhibited moderate (˜10%) editing efficiency. The underlying cause might be the inefficient dimerization driven by RNA-RNA interaction, or possible degradation of RNA duplex that lies outside of the Cas9-gRNA complex, unprotected from RNA endonuclease degradation. In the previous report, the tethering of trans prime editing RNA to Prime Editor protein using MS2-MCP interaction exhibited higher editing efficiency⁷. However, this report also revealed that physical tethering between gRNA and trans-prime-editing RNA is not necessary for inducing prime editing; prime editing can be completely split into two independent modules, where sgRNA-Cas9 complex nicks the target and RTase-petRNA complex reverse-transcribes off of the nicked strand. Therefore, this approach is not suitable because we aim to record specific interactions between two molecules.

Lastly, we tested the impact of inserting self-splicing ribozymes within the pegRNA to prime editing efficiency. We tested 6 sites within pegRNA to insert the whole ribozyme sequence⁵ (413-bp in length). We observed low editing efficiency (<2%) using pegRNAs containing the ribozyme sequence. We also tested the insertion of deactivated ribozyme In two positions, which showed a greater than a 10-fold decrease in the editing efficiency. This suggests that the prime editing depends on the ribozyme function, but the insertion of the ribozyme sequence renders the pegRNA less active, possibly due to a low self-splicing efficiency by the ribozyme in the context of pegRNA. We reasoned that insertion of ribozyme substantially reduces the activity of pegRNA, and decided not to pursue testing the split-ribozyme, which may reduce the editing efficiency further.

In summary, we have tested three strategies to engineer Split-pegRNA recorders, which revealed that the splitting pegRNA at the repeat-antirepeat portion is the most promising strategy. The genome editing efficiencies matched the RNA-RNA interaction strength between repeat and antirepeat sequences, demonstrating that we can record RNA-RNA interaction strengths to the genome via prime editing. Our design mimics the functional molecules within the CRISPR-Cas9 system. Therefore, we pursued this strategy for designing Split-pegRNA recorder constructs, which we referred to as crRNA and petracrRNA herein.

### Designing Programmable Dimerization Domains on Split-pegRNA

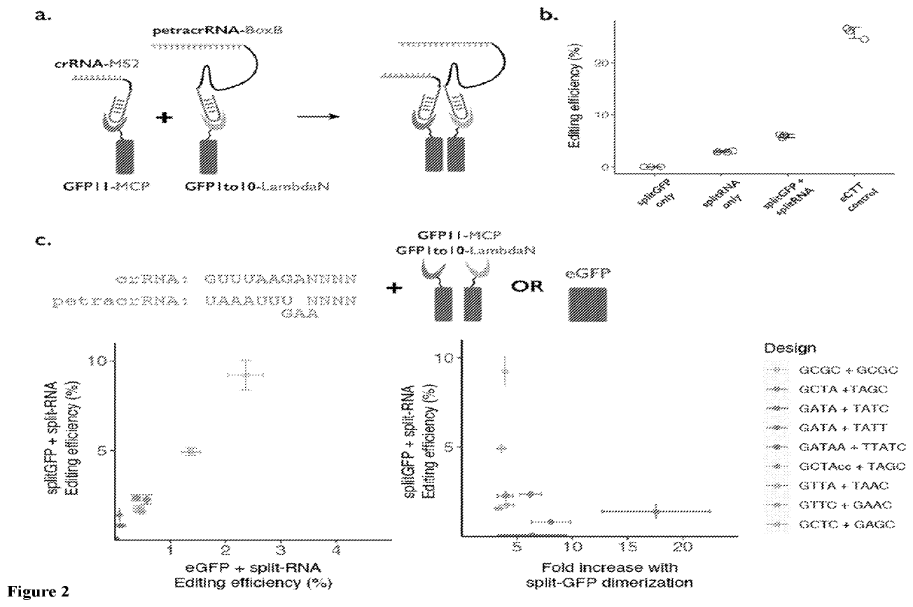

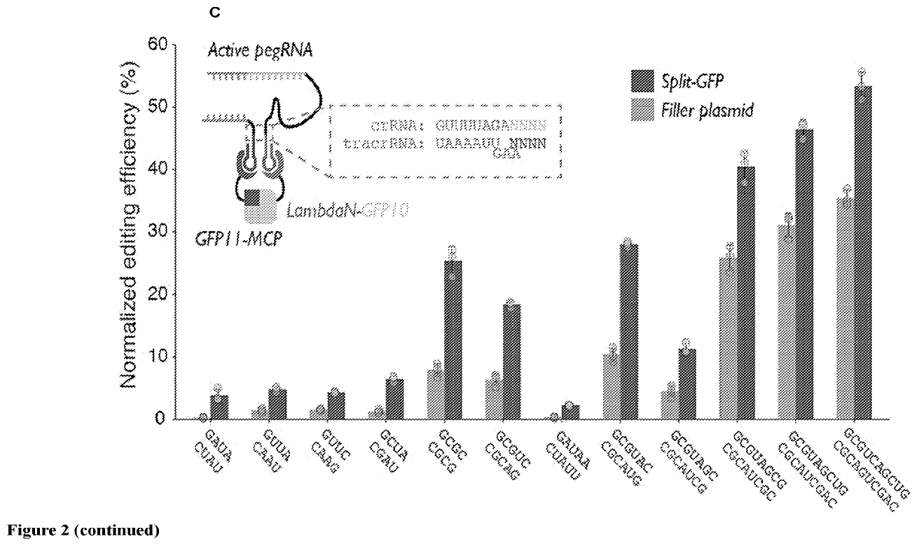

The repeat-antirepeat sequences, respectively in crRNA and petracrRNA, include a portion that is necessary for binding the Cas9 molecule (FIG. 1c) and an extra portion that can be removed without affecting the Cas9 function within the joined sgRNA (FIG. 1c). The latter domain effectively serves as an RNA-RNA hetero-dimerization module; its removal reduces the editing efficiency (FIG. 1d,e). We reasoned that this portion could be replaced by a protein-protein hetero-dimerization module by installing the two orthogonal protein-RNA binding adaptors (i.e., MS2 and BoxB RNA stem-loops that specifically bind to MCP and lambdaN proteins respectively) (FIG. 2a). In theory, physical proximity between two interacting protein molecules, each stably tethered to crRNA and petracrRNA, may induce the formation of functional pegRNA.

To test the proposed layout for recording protein-protein interaction, we chose the split-GFP system that includes two components, GFP1-10 and GFP11, which dimerize to form functional fluorescent proteins.

GFP1-10 amino-acid sequence
(SEQ ID NO: 112)
EFMSKGEELFTGVVPILVELDGDVNGHKFSVRGEGEGDATIGKLTLKFI
CTTGKLPVPWPTLVTTLTYGVQCESRYPDHMKRHDFFKSAMPEGYVQER
TISFKDDGKYKTRAVVKFEGDTLVNRIELKGTDFKEDGNILGHKLEYNE
NSHNVYITADKQKNGIKANFTVRHNVEDGSVQLADHYQQNTPIGDGPVL
LPDNHYLSTQTVLSKDPNEKGT
GFP11 amino-acid sequence
(SEQ ID NO: 113)
MRDHMVLHESVNAAGIT

We appended both molecules with adaptor modules, lambdaN (λN) and MCP proteins, which tightly bind to RNA structures, BoxB and MS2 stem-loops, respectively. We also included BoxB and MS2 sequences to petracrRNA and crRNA, respectively, replacing the previous RNA-RNA dimerization modules. We designed and cloned three constructs: 1) U6::crRNA-MS2, expressing crRNA with MS2 stem-loop that binds to MCP protein (SEQ ID NO: 36), where U6 is the promoter used in the construct (not included in SEQ ID NO:36 sequence), 2) U6::petracrRNA-BoxB, expressing pe-tracrRNA with BoxB stem-loop that binds to λN protein (SEQ ID NO:37), and 3) pCMV::GFP11-NLS-MCP-IRES-NLS-λN-GFP1-10 (SEQ ID NO:38), a polycistronic expression cassette for two proteins based on the split-GFP system which we refer to as “Split-GFP-RNAadaptor” protein pairs. Dimerization of GFP1-10 to GFP11 to form a functional GFP molecule would bring MCP-bound crRNA-MS2 and λN-bound pe-tracrRNA-BoxB close, possibly forming a functional prime editing complex with the prime editor protein. We have included several nuclear localization sequences (NLS) within the protein adaptors because the protein-protein interaction will be localized within the nucleus where prime editing occurs. For example, see SEQ ID NO:38-47, which include an encoded NLS. Any nuclear localization sequences can be used, including but not limited to BpNLS_SV40: KRTADGSEFESPKKKRKV (SEQ ID NO:130), C-Myc-NLS: PAAKRVKLD (SEQ ID NO:131), and KRPAATKKAGQAKKKK (SEQ ID NO: 132).

Upon transfection of Split-GFP-RNAadaptor plasmid, we observed positive GFP signals that are specific to the cell nucleus of HEK293T, distinguishable from GFP signals lacking the NLS element, suggesting that split-GFP dimerization occurs within the nucleus as intended. Transfection of three plasmid constructs described above along with the Prime Editor expressing plasmid (pCMV-PEmax-P2A-hMLH1dn or PE4max) (SEQ ID NO:39) induced the genome editing programmed by the petracrRNA (FIG. 2b). Without the split-GFP plasmid, however, we still observed a substantial editing efficiency, which is a background signal driven by the dimerization of crRNA and petracrRNA without the dimerization of split-GFP. The level of background editing was similar to crRNA/petracrRNA constructs without MS2/BoxB modules (Design 2 in FIG. 1c), consistent with our hypothesis of the source of background as split-GFP independent dimerization.

To improve the signal-to-noise ratio in our recording of protein-protein dimerization, we designed multiple pairs of crRNA-petracrRNA by altering the top four base pairs within the repeat-antirepeat loop of guide RNA. In all 12 pairs, we observe that presence of Split-GFP-RNAadaptor increases the editing efficiency of crRNA-petracrRNA, possibly by increasing the formation of functional pegRNA (FIG. 2c). We also observe that stronger base-pairing in the top four base pairs (i.e., higher GC contents) increases both the editing efficiency and the background editing level, indicating that the editing depends on the dimerization of crRNA and petracrRNA components (SEQ ID NO:84-107).

Recording Protein-Protein Dimerization Events within the Cell

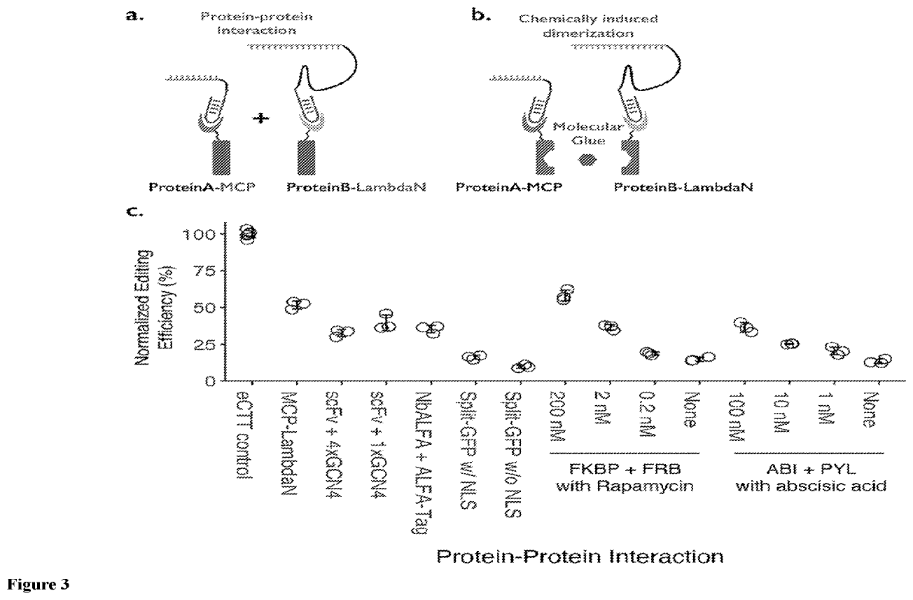

We tested recording of different protein-protein interactions using the crRNA-MS2 (SEQ ID NO:36) and petracrRNA-BoxB (SEQ ID NO:37) constructs with “GCGC” design as described above. To account for differences in editing efficiency in multiple independent rounds of testing prime editing, we decided to calculate and compare “normalized editing efficiencies”, which we calculated by dividing the observed editing efficiency by positive control editing efficiency (insertion of the same CTT to the native HEK3 target using epegRNA-expressing plasmid, rather than crRNA and petracrRNA, usually achieving the editing efficiency of 25-40%). To test the editing of crRNA-MS2/petracrRNA-BoxB complex, we transfected a single construct of MCP domain directly tethered to lambdaN domain (SEQ ID NO:133), which showed a high editing efficiency (normalized editing efficiency of 50% compared to standard epegRNA) (FIG. 3c). Next, we tested a protein dimerization of SUN-Tag system (scFv domain binding to GCN4 epitope) and ALFA-Tag system (ALFA-nanobody or NbALFA binding to ALFA-tag of 15 amino-acids), which showed a range of 30 to 40% normalized editing efficiencies. We observed that the addition of GCN4 epitope tags did not substantially increase the editing efficiency, a common strategy to increase the signal of scFv binding in SUN-Tag system.

scFv amino-acid sequence
(SEQ ID NO: 114)
MGPDIVMTQSPSSLSASVGDRVTITCRSSTGAVTTSNYASWVQEKPGKL
FKGLIGGINNRAPGVPSRFSGSLIGDKATLTISSLQPEDFATYFCALWY
SNHWVFGQGTKVELKRGGGGSGGGGSGGGGSSGGGSEVKLLESGGGLVQ
PGGSLKLSCAVSGFSLTDYGVNWVROAPGRGLEWIGVIWGDGITDYNSA
LKDRFIISKDNGKNTVYLOMSKVRSDDTALYYCVTGLEDYWGQGTLVTV
SS*
GCN4 amino-acid sequence
(SEQ ID NO: 115)
EELLSKNYHLENEVARLKK
NbALFA amino-acid sequence
(SEQ ID NO: 116)
SGEVQLQESGGGLVQPGGSLRLSCTASGVTISALNAMAMGWYRQAPGER
RVMVAAVSERGNAMYRESVQGRFTVTRDFTNKMVSLQMDNLKPEDTAVY
YCHVLEDRVDSFHDYWGQGTQVTVSSGAPGESSISA*
ALFA amino-acid sequence
(SEQ ID NO: 117)
PSRLEEELRRRLTEP
(SEQ ID NO: 133)
MEQKLISEEDLKRPAATKKAGQAKKKKGGSASGGSMDAQTRRRERRAEK
QAQWKAANSGGSSGGSKRTADGSEFESPKKKRKVSGGSSGGSGGPAAKR
VKLDGSGASNFTQFVLVDNGGTGDVTVAPSNFANGVAEWISSNSRSQAY
KVTCSVRQSSAQKRKYTIKVEVPKVATQTVGGVELPVAAWRSYLNMELT
IPIFATNSDCELIVKAMQGLLKDGNPIPSAIAANSGLY

In the tagged protein constructs, we have included nuclear localization sequence (NLS) to ensure the localization of the protein pairs in the nucleus, where the genome editing takes place. To understand whether the localization of dimer into the nucleus via NLS is the necessary component, we have cloned another construct without NLSs to localize the GFP1-10 and GFP11 within the nucleus. Using microscopy, we have confirmed that the GFP signal is present across the cell nucleus and cytoplasm, compared to sharp localization within the nucleus in the construct with NLSs. We also observe a slight drop in the editing efficiency (FIG. 3c), which could be due to the diluted concentration of splitGFP-crRNA-petracrRNA complex across the cell rather than concentrated within the nucleus. We envision that existing biosensors that take advantage of induced translocation events from membrane/cytoplasm to nucleus can be used in this system, where specific cellular events allow crRNA-petracrRNA to translocate to the nucleus for editing.

Recording Small Molecules that Induce Protein Hetero-Dimerization

In the past 30 years, several chemicals have been identified as critical signaling molecules for promoting protein-protein interactions⁸. We reasoned that the exposure of these “molecular glue” small molecules to cells can be recorded into the genome using specifically engineered split-pegRNA recorders. To demonstrate the recording of past small molecule exposure, we chose Rapamycin-induced dimerization of FKBP (FK506 Binding Proteins) (SEQ ID NO:45, fused to MCP) and FRB protein domains (SEQ ID NO:44; fused to Lambda N) of the m TOR pathway and Abscisic acid (ABA)-induced dimerization of pyrabactin resistance domain (PYL) (SEQ ID NO:43; fused to MCP) and ABA-insensitive (ABI) domain (SEQ ID NO:42; fused to Lambda N) (FIG. 3b). We observed a strong increase in the editing efficiency of each FKBP-FRB and PYL-ABI pairs in the addition of small molecules that induce dimerization (FIG. 3c).

PYL amino-acid sequence
(SEQ ID NO: 118)
GAPTQDEFTQLSQSIAEFHTYQLGNGRCSSLLAQRIHAPPETVWSVVRR
FDRPQIYKHFIKSCNVSEDFEMRVGCTRDVNVISGLPANTSRERLDLLD
DDRRVTGFSITGGEHRLRNYKSVTTVHRFEKEEEEERIWTVVLESYVVD
VPEGNSEEDTRLFADTVIRLNLQKLASITEAMN
ABI amino-acid sequence
(SEQ ID NO: 119)
TRVPLYGFTSICGRRPEMEAAVSTIPRFLOSSSGSMLDGREDPQSAAHF
FGVYDGHGGSQVANYCRERMHLALAEEIAKEKPMLCDGDTWLEKWKKAL
FNSFLRVDSEIESVAPETVGSTSVVAVVFPSHIFVANCGDSRAVLCRGK
TALPLSVDHKPDREDEAARIEAAGGKVIQWNGARVFGVLAMSRSIGDRY
LKPSIIPDPEVTAVKRVKEDDCLILASDGVWDVMTDEEACEMARKRILL
WHKKNAVAGDASLLADERRKEGKDPAAMSAAEYLSKLAIQRGSKDNISV
VVVDLK

### Discussion

We have developed the Split-pegRNA recording systems, where RNA-RNA and protein-protein interactions can be converted to molecular recordings on DNA medium within living cells. In addition to our demonstrated use described in this manuscript, we also imagine the adaptation of our approach to recording wider molecular events within the cell. First, by replacing the RNA-protein adaptor with another RNA structure of interest, RNA-protein interaction can be measured in our recording assay. Our approach could be used to screen RNA-RNA, RNA-protein, and protein-protein heterodimerization strength in a massively-parallel approach, revealing how mutations in RNA or protein affect its binding to partners in the cellular context, as opposed to in vitro purified and reconstituted system. The key to uncovering the underlying energy landscape in biomolecular interaction can be a calibration of recording efficiency with other measurements such as dissociation constant (K_{D}) between two macromolecules.

In addition, one could also install a strong protein-protein dimerization module (e.g., Sun Tag system) to detect the expression of protein levels. For example, one could constitutively express crRNA-MS2, BoxB-petracrRNA, MCP-scFv protein, and tag endogenous or cargo protein with AN-GCN4 epitope molecule, where GCN4 epitope will strongly bind to scFv. The expression of the target protein will result in the formation of functional pegRNA to record its occurrence and strength. Using existing systems such as RADAR and transcription cis-regulatory elements, RNA expression, and transcriptional activity can be recorded using the Split-pegRNA recording systems, respectively.

One of the most well-known information transfer systems in biology is the expression of protein from the genome. In this case, the genomic DNA serves as an information retrieval medium, in which the necessary information of the amino-acid sequence for functional protein is encoded in the DNA sequence. The information process is bridged by RNA through the regulation of its expression and function. Both transcription and translation also serve to amplify the genetic signal, where many copies of RNA transcripts are produced from a single DNA molecule, and many copies of protein molecules are synthesized from a single RNA transcript molecule. In the present recording system, the information flow is reversed, where the molecular events are sensed by interactions at the protein and RNA level, and transferred to DNA sequences as an information recording medium. Therefore, we envision a complete circular system, where the protein expression, RNA modulation, and DNA editing system can evolve once it is introduced into a living cell, sensing cellular environments to modify specific genetic circuits encoded in DNA, which expresses proteins to alter its cellular environment.

### Materials and Methods

### Plasmid Cloning

All crRNA and petracrRNA constructs were cloned using ligation after restriction (T4 DNA Ligase, New England Biolabs), following the protocol outlined in Anzalone et al.¹. Single-stranded DNAs (IDT) were annealed to have 4 bp overhangs in both ends of double-stranded DNAs, which is a substrate for T4 DNA ligase. The plasmid backbone (pU6-pegRNA-GG-acceptor, Addgene #132777) was digested using BsaI-HFv2, and mixed with annealed double-stranded DNA constructs with 4-bp overhangs. At the end of all crRNA and petracrRNA constructs, we added the evoPreQ1 sequence and poly-T terminator sequence. A small amount (1-2 uL) of T4 ligation reaction mix was added to NEB Stbl cell (C3040) for transformation and grown at 37° C. for the plasmid DNA preparation (Qiagen miniprep). The resulting plasmids were sequence-verified using Sanger sequencing (Genewiz).

All protein expression constructs were cloned using Gibson assembly (NEB, where double-stranded DNA fragments are either ordered from IDT as gBlocks or PCR amplified from existing constructs with at least 25-bp overlap in sequence. A small amount (1-2 uL) of Gibson Assembly reaction mix was added to NEB Stbl cell (C3040) for transformation and grown at 30° C. or 37° C. for the plasmid DNA preparation (Qiagen miniprep). The resulting plasmids were sequence-verified using Sanger sequencing (Genewiz).

### Tissue Culture, Transfection, Lentiviral Transduction, and Transgene Integration

The HEK293T cell line was purchased from ATCC, and maintained by following the recommended protocol from the vendor. HEK293T cells were cultured in Dulbecco's modified Eagle's medium (DMEM) with high glucose (GIBCO), supplemented with 10% fetal bovine serum (Rocky Mountain Biologicals) and 1% penicillin-streptomycin (GIBCO). Cells were grown with 5% CO₂ at 37° C. Cell lines were used as received without authentication or a test for Mycoplasma.

For transient transfection, HEK293T cells were cultured to 70-90% confluency in a 24-well plate. For prime editing with crRNA/petracrRNA, 375 ng of PE4max enzyme plasmid (Addgene #174828), 62.5 ng of crRNA plasmid, and 62.5 ng of petracrRNA plasmid were mixed and prepared with a transfection reagent (Lipofectamine™ 3000) following the recommended protocol from the vendor. For Split-GFP-RNAadaptor or Split-FKBP-RNAadaptor™ experiments, 250 ng of PE4max enzyme plasmid, 125 ng of Split-GFP-RNAadaptor™ or Split-FKBP-RNAadaptor™ plasmid, 62.5 ng of crRNA plasmid, and 62.5 ng of petracrRNA plasmid were used in transfection. Cells were cultured for four days after the initial transfection unless noted otherwise, and their genomic DNA was harvested following cell lysis and protease protocol from Anzalone et al.

### Genomic DNA Collection and Sequencing Library Preparation

The targeted region from collected genomic DNA was amplified using two-step PCR and sequenced using the Illumina sequencing platform (NextSeq™ or MiSeq™). The first PCR reaction (KAPA Robust polymerase) included 1.5 uL of cell lysate, 0.04 to 0.4 uM of forward and reverse primers in a final reaction volume of 25 uL. We programmed the first PCR reaction to be: (1) 3 minutes at 95° C., (2) 15 seconds at 95° C., (3) 10 seconds at 65° C., (4) 90 seconds at 72° C., (5) 25-28 cycles of repeating step 2 through 4, and (6) 1 minute at 72° C. Primers included sequencing adapters to their 3′-ends, appending them to both termini of PCR products that amplified genomic DNA. After the first PCR step, products were assessed on 6% TBE-gel and purified using 1.0×AMPure™ (Beckman Coulter) and added to the second PCR reaction that appended dual sample indexes and flow cell adapters. The second PCR reaction program was identical to the first PCR program except we ran it for only 5-10 cycles. Products were again purified using AMPure and assessed on the TapeStation (Agilent) before being denatured for the sequencing run.

### Genomic DNA Amplicon Sequencing Data Processing and Analysis

Sequencing reads from Illumina MiSeq™ and NextSeq™ platforms are first demultiplexed using BCL2fastq software (Illumina). Sequencing libraries were single-end sequenced to cover the DNA Tape from one direction. Editing efficiencies were calculated using pattern-matching software such as Regular Expression (package REGEX) in Python, counting correct amplicon reads with or without intended edits (CTT insertion to HEK3 locus¹).

### REFERENCES

1. Anzalone, A. V. et al. Search-and-replace genome editing without double-strand breaks or donor DNA. Nature 576, 149-157 (2019).
2. Choi, J. et al. A temporally resolved, multiplex molecular recorder based on sequential genome editing. bioRxiv 2021.11.05.467388 (2021) doi:10.1101/2021.11.05.467388.
3. Chen, W. et al. Multiplex genomic recording of enhancer and signal transduction activity in mammalian cells. bioRxiv 2021.11.05.467434 (2021) doi:10.1101/2021.11.05.467434.
4. Jinek, M. et al. A Programmable Dual-RNA-Guided DNA Endonuclease in Adaptive Bacterial Immunity. Science 337, 816-821 (2012).
5. Gambill, L., Staubus, A., Ameruoso, A. & Chappell, J. A split ribozyme that links detection of a native RNA to orthogonal protein outputs. bioRxiv 2022.01.12.476080 (2022) doi:10.1101/2022.01.12.476080.
6. Nelson, J. W. et al. Engineered pegRNAs improve prime editing efficiency. Nat. Biotechnol. (2021) doi:10.1038/s41587-021-01039-7.
7. Liu, B. et al. A split prime editor with untethered reverse transcriptase and circular RNA template. Nat. Biotechnol. (2022) doi:10.1038/s41587-022-01255-9.
8. Schreiber, S. L. The Rise of Molecular Glues. Cell 184, 3-9 (2021).
9. Eerik Kaseniit, K. et al. Modular and programmable RNA sensing using ADAR editing in living cells. bioRxiv 2022.01.28.478207 (2022) doi:10.1101/2022.01.28.478207.
10. Jiang, K. et al. Programmable eukaryotic protein expression with RNA sensors. bioRxiv 2022.01.26.477951 (2022) doi:10.1101/2022.01.26.477951.

1. A split pegRNA comprising:
(a) a crRNA component, comprising in 5′ to 3′ order:
(i) a spacer sequence for a genomic locus of interest;
(ii) a crRNA repeat sequence necessary for binding to a CRISPR-Cas protein; and
(iii) a first RNA extension; and
(b) a petracrRNA component, comprising in 5′ to 3′ order:
(i) a second RNA extension;
(ii) a petracrRNA antirepeat sequence necessary for binding to a CRISPR-Cas protein;
(iii) a gRNA scaffold;
(iv) an optional reverse transcriptase template sequence (RTS); and
(v) an optional primer binding site (PBS);
wherein the crRNA component and the petracrRNA component are not covalently bound to each other;
wherein:
(I) the first RNA extension is capable of binding to a first protein, and the second RNA extension is capable of binding to a second protein, wherein the first protein and the second protein are capable of binding to each other; or
(II) wherein one domain of the first protein and the first RNA extension are capable of binding to each other, and another domain of the first protein and the second RNA extension are capable of binding to each other; or
(III) the first RNA extension is capable of binding to a first protein, and the second RNA extension is capable of binding to a second protein, wherein there exists a chemical that induce the binding between the first protein and the second protein when introduced to cells or naturally synthesized within cells.

2. The split pegRNA of claim 1, wherein the CRISPR-Cas protein is selected from the group consisting of Cas9, nickases, nucleases, deactivated Cas9, modified Cas9 in Base Editors, and CRISPR-Cas proteins used with other epigenetic effector modules, e.g. CRISPRa/i.

3. (canceled)

4. The split pegRNA of claim 1, wherein the first RNA extension is capable of binding to a first protein, and the second RNA extension is capable of binding to a second protein, wherein the first protein and the second protein are capable of binding to each other.

5. The split pegRNA of claim 1, wherein one of the first and second RNA extensions comprises an MS2 RNA stem loop, BoxB RNA stem loop, PP7 RNA stem loop, or HIV-1 TAR stem loop, and the other comprises any other RNA stem loop with a known stable protein binding partner, or wherein one first and second RNA extensions comprises an MS2 RNA stem loop, and the other comprises a BoxB RNA stem loop, or wherein one of the first and second RNA extensions comprises a PP7 RNA stem loop, and the other comprises an HIV-1 TAR stem loop.

6.-7. (canceled)

8. The split pegRNA of claim 4, wherein
(a) the MS2 RNA stem loop comprises or consists of the nucleic acid sequence selected from the group consisting of SEQ ID NO:1-2; and
(b) the BoxB RNA stem loop comprises or consists of the nucleic acid sequence of SEQ ID NO:3-4;
wherein optional residues may be present or may be deleted; or
wherein the PP7 RNA stem loop comprises or consists of the nucleic acid sequence: of SEQ ID NO:5, and the HIV-1 TAR stem loop comprises or consists of the nucleic acid sequence of SEQ ID NO:6.

9. (canceled)

10. The split pegRNA of claim 1, wherein one domain of the first protein and the first RNA extension are capable of binding to each other, and another domain of the first protein and the second RNA extension are capable of binding to each other, either constitutively or dynamically controlled by additional chemicals that induce binding, or wherein the first protein comprises MCP domain for binding MS2 RNA hairpin, LambdaN domain for binding BoxB RNA hairpin, PCP domain for binding PP7 RNA hairpin, or HIV-1 Tat domain for binding TAR RNA hairpin.

11. (canceled)

12. The split pegRNA of claim 1, wherein the crRNA repeat comprises or consists of the nucleic acid sequence selected from the group consisting of SEQ ID NO:7-19, and/or wherein the petracrRNA scaffold comprises or consists of the nucleic acid sequence selected from the group consisting of SEQ ID NO:20-32, and/or wherein the gRNA scaffold comprises or consists of a nucleic acid sequence at least 25%, 30% 40%, 50%, 60%, 70%, 80%, 90%, 95%, 98%, or 100% identical to the nucleic acid sequence of SEQ ID NO:33.

13.-14. (canceled)

15. The split pegRNA of claim 1, wherein the first RNA extension is capable of binding to a first protein, and the second RNA extension is capable of binding to a second protein, wherein there exists a chemical that induces the binding between the first protein and the second protein when introduced to cells or naturally synthesized within cells.

16. The split pegRNA of claim 15, wherein the chemical is a small molecule selected from rapamycin and abscisic acid.

17. (canceled)

18. The split pegRNA of claim 16, wherein the small molecule is rapamycin, and wherein the first protein and the second protein comprise FKBP (FK506 Binding Proteins) and FRB (FKBP-Rapamycin Binding), optionally wherein the first and second proteins comprise fusion proteins with MCP domain for binding MS2 RNA hairpin, LambdaN domain for binding BoxB RNA hairpin, PCP domain for binding PP7 RNA hairpin, or HIV-1 Tat domain.

19. (canceled)

20. The split pegRNA of claim 164, wherein the small molecule is abscisic acid (ABA)- and the first protein and the second protein comprise pyrabactin resistance domain (PYL) and ABA-insensitive (ABI) domain, optionally wherein the first and second proteins comprise fusion proteins with MCP domain for binding MS2 RNA hairpin, LambdaN domain for binding BoxB RNA hairpin, PCP domain for binding PP7 RNA hairpin, or HIV-1 Tat domain.

21.-22. (canceled)

23. The split pegRNA of claim 1, wherein the crRNA component and/or the petracrRNA component further comprises an RNA stabilization domain at their 3′ terminus, optionally wherein the crRNA component and the petracrRNA component further comprises an RNA stabilization domain at their 3′ terminus, optionally wherein the RNA stabilization domain comprises or consists of the nucleic acid sequence of SEQ ID NO: 35.

24.-25. (canceled)

26. The split pegRNA of claim 1, wherein the crRNA component comprises the genus X1-X2, wherein X1 is a spacer sequence for a genomic locus of interest, and X2 comprises or consists of a nucleic acid sequence of the formula B1-B2-B3, wherein B1, B2, and B3, respectively, comprise or consist of, in 5′ to 3′ order:
SEQ ID NO:7-SEQ ID NO:2-SEQ ID NO:35;
SEQ ID NO:8-SEQ ID NO:2-SEQ ID NO:35;
SEQ ID NO:9-SEQ ID NO:2-SEQ ID NO:35;
SEQ ID NO:10-SEQ ID NO:2-SEQ ID NO:35;
SEQ ID NO:11-SEQ ID NO:2-SEQ ID NO:35;
SEQ ID NO:12-SEQ ID NO:2-SEQ ID NO:35;
SEQ ID NO:13-SEQ ID NO:2-SEQ ID NO:35;
SEQ ID NO:14-SEQ ID NO:2-SEQ ID NO:35;
SEQ ID NO:15-SEQ ID NO:2-SEQ ID NO:35;
SEQ ID NO:16-SEQ ID NO:2-SEQ ID NO:35;
SEQ ID NO:17-SEQ ID NO:2-SEQ ID NO:35;
SEQ ID NO:18-SEQ ID NO:2-SEQ ID NO:35; or
SEQ ID NO: 19-SEQ ID NO:2-SEQ ID NO:35; optionally wherein X1 is a spacer sequence for a genomic locus of interest, and X2 comprises or consists of the nucleic acid sequence of SEQ ID NO:36.

27. (canceled)

28. The split pegRNA of claim 1, wherein the petracrRNA component comprises a nucleic acid sequence of the genus Z1-Z2-Z3-Z4-Z5, wherein
Z1 comprises or consists of a BoxB nucleotide sequence selected from the group consisting of SEQ ID NO:3-4;
Z2 comprises or consists of a petracrRNA antirepeat nucleotide sequence selected from the group consisting of SEQ ID NO:20-32;
Z3 comprises a gRNA scaffold
Z4 comprises an RTT and PBS sequence, or is absent; and
Z5 comprises an RNA stabilization sequence or is absent; optionally wherein Z5 is present and comprises or consists of the RNA stabilization sequence of SEQ ID NO:35; optionally wherein Z3 comprises or consists of a nucleic acid sequence at least 25%, 30%, 40%, 50%, 60%, 70%, 80%, 90%, 95%, 98%, or 100% identical to the gRNA scaffold nucleotide sequence of SEQ ID NO:33.

29.-30. (canceled)

31. The split pegRNA of claim 1, wherein the petracrRNA component comprises or consists of a nucleic acid sequence of the formula O1-O2-O3-O4, wherein O1, O2, O3, and O4, respectively, comprise or consist of, in 5′ to 3′ order:
SEQ ID NO:4-SEQ ID NO:21-SEQ ID NO:33-SEQ ID NO:35;
SEQ ID NO:4-SEQ ID NO:22-SEQ ID NO:33-SEQ ID NO:35;
SEQ ID NO:4-SEQ ID NO:23-SEQ ID NO:33-SEQ ID NO:35;
SEQ ID NO:4-SEQ ID NO:24-SEQ ID NO:33-SEQ ID NO:35;
SEQ ID NO:4-SEQ ID NO:25-SEQ ID NO:33-SEQ ID NO:35;
SEQ ID NO:4-SEQ ID NO:26-SEQ ID NO:33-SEQ ID NO:35;
SEQ ID NO:4-SEQ ID NO:27-SEQ ID NO:33-SEQ ID NO:35;
SEQ ID NO:4-SEQ ID NO:28-SEQ ID NO:33-SEQ ID NO:35;
SEQ ID NO:4-SEQ ID NO:29-SEQ ID NO:33-SEQ ID NO:35;
SEQ ID NO:4-SEQ ID NO:30-SEQ ID NO:33-SEQ ID NO:35;
SEQ ID NO:4-SEQ ID NO:31-SEQ ID NO:33-SEQ ID NO:35; or
SEQ ID NO:4-SEQ ID NO:32-SEQ ID NO:33-SEQ ID NO:35; optionally wherein the petracrRNA component comprises or consists of the nucleic acid sequence of SEQ ID NO:37.

32. (canceled)

33. A nucleic acid encoding the crRNA as recited in claim 1.

34. A nucleic acid encoding the petracrRNA as recited in claim 1.

35. An expression vector comprising the nucleic acid of claim 33 linked to a suitable control element, such as a promoter, optionally wherein the expression vector is present in a host cell.

36. (canceled)

37. A kit, comprising the split-pegRNA of claim 1.

38.-39. (canceled)

40. A method for recording a protein-protein and/or protein-RNA interaction within a cell, comprising expressing in the cell:
(a) the split pegRNA of claim 1,
(b) the first and, if necessary, the second protein of claim 1, and
(c) the modified or unmodified CRISPR-Cas protein as recited in claim 1;
wherein dimerization of the first and second RNA extensions of the split pegRNA via binding to the first and, if necessary, the second protein in the cell induces formation of functioning guide RNA for genome or epigenome editing via the CRISPR-Cas protein, thereby inducing genome or epigenome editing to edit genomic DNA in the cell and recording of the protein-protein and/or protein-RNA interaction into genomic DNA in the cell; or
wherein dimerization of the first and second RNA extensions of the split pegRNA via binding to the first and the second protein in the cell, which is controlled by chemicals that induce binding between the first and second proteins, induces formation of functioning guide RNA for genome or epigenome editing via the CRISPR-Cas protein, thereby controlling genome or epigenome editing to edit genomic DNA in the cell with chemicals that control protein-protein dimerization.

41. (canceled)
